(19) United States
(12) Patent Application Publication
(10) Pub. No.: US 2022/0088016 A1
(43) Pub. Date: Mar. 24, 2022

(54) CANCER TREATMENT USING MULTI-TARGETED KINASE INHIBITOR IN COMBINATION OF PROTEIN KINASE BIOMARKERS

(71) Applicant: ZHEJIANG CROWNMAB BIOTECH CO. LTD., Hangzhou (CN)

(72) Inventors: Lan YANG, Hangzhou (CN); Qian SHI, Hangzhou (CN); Guoliang YU, Hangzhou (CN)

(21) Appl. No.: 17/420,391

(22) PCT Filed: Jan. 2, 2020
(86) PCT No.: PCT/CN2020/070041

§ 371 Date: Jul. 2, 2021

(30) Foreign Application Priority Data
Jan. 2, 2019 (CN) PCT/CN2019/070041

(51) Int. Cl. A61K 31/505; A61P 35/00; G01N 33/574

(52) U.S. Cl. CPC A61K 31/505; G01N 2333/912; G01N 33/574; A61P 35/00

(57) ABSTRACT

Provided herein are methods of treating cancer in a patient with a tyrosine kinase inhibitor. The method comprises: a) measuring the expression level of a protein kinase in a sample obtained from the patient; b) comparing the expression level of the protein kinase to a corresponding reference expression level; c) determining a likelihood of the patient being responsive to the tyrosine kinase inhibitor; and d) treating the patient whose expression level of the protein kinase indicates that the patient will be responsive with the tyrosine kinase inhibitor.

### CROSS-REFERENCE TO RELATED APPLICATIONS

This application claims priority to PCT/CN2019/070041, filed Jan. 2, 2019, the disclosure of which is incorporated herein by reference.

### SEQUENCE LISTING

The sequence listing that is contained in the file named “071017-8006W002-SL-20200102 ST25”, which is 26 KB (as measured in Microsoft Windows) and was created on Jan. 2, 2020, is filed herewith by electronic submission and is incorporated by reference herein.

### FIELD OF THE INVENTION

The present invention generally relates to cancer treatment. In particular, the present invention relates to methods for treating a cancer using a multi-targeted kinase inhibitor in combination of protein kinase biomarkers.

### BACKGROUND

Multi-targeted tyrosine kinase inhibitors have played an increasingly important role in the treatment of cancer, such as non-small cell lung cancer (NSCLC) (see, e.g., Mok T S, et al. N Engl J Med (2009) 361:947-57). The promising anti-tumor effect of multi-targeted tyrosine kinase is based on the theory that single-targeted drugs often have poor efficacy because cancer is usually a heterogenous malignancy. When blocking the key signal transduction pathways, the single-targeted drugs can also activate the tumor escape mechanisms. As a result, tumor cell proliferation can be re-activated through other pathways. Therefore, drugs should be optimized to inhibit as many as possible of the tumor signal pathway.

Despite of the theoretical advantage, many clinical trials of multi-targeted tyrosine kinase inhibitors did not show satisfactory results (see Zhou C, Transl Lung Cancer Res (2012) 1:72-77). Therefore, there is a need to design and develop therapeutic regime to improve the efficacy of treatment using multi-targeted tyrosine kinase inhibitors.

### SUMMARY

In one aspect, the present disclosure provides a method for treating cancer in a patient with a tyrosine kinase inhibitor. In one embodiment, the method comprises: a) measuring the expression level of a first protein kinase in a sample obtained from the patient; b) comparing the expression level of the first protein kinase to a corresponding reference expression level; c) determining a likelihood of the patient being responsive to the tyrosine kinase inhibitor; and d) treating the patient whose expression level of the first protein kinase indicates that the patient will be responsive with the tyrosine kinase inhibitor.

In certain embodiments, the first protein kinase is selected from the group consisting of DDR1, CSF1R, CDKL2, cKit, c-RAF, Flt1, Flt4, KDR, MAP4K5, PDGFRα, PTK5, Ret, SAPK2b and ZAK. In certain embodiments, the first protein kinase contains a mutation. In one embodiment, the mutation is cKit(V560G) or PDGFRα(V561D). In one embodiment, the first protein kinase is DDR1 or CSF1R.

In certain embodiments, the tyrosine kinase inhibitor is a multi-targeted tyrosine kinase inhibitor. In some embodiments, the multi-targeted tyrosine kinase inhibitor preferentially inhibits a second protein kinase different from the first protein kinase. In yet another embodiment, the second protein kinase is KDR.

In certain embodiments, the tyrosine kinase inhibitor is an antibody, an antisense oligonucleotide or a compound. In some embodiments, the tyrosine kinase inhibitor is a compound of formula (I) or a pharmaceutically acceptable salt thereof

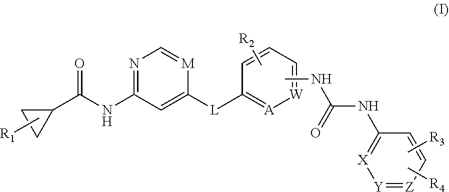

wherein:
R₁ is hydrogen, C₁₋₆ alkyl, C₁₋₆ haloalkyl, halo or cyano;
M is CH or N;
L is O, NH or N(CH₃);
A is CR₅ or N;
W is CR₆ or N;
R₂, R₅ and R₆ are independently hydrogen, C₁₋₆ alkyl, C₁₋₆ haloalkyl, halo, C₃₋₇ cycloalkyl or cyano;
X, Y, and Z are independently CH or N; and
R₃ and R₄ are independently hydrogen, halo, cyano, C₁₋₆ alkyl, C₁₋₆ haloalkyl, C₂₋₆ alkenyl, hydroxyl-C₁₋₆ alkyl, di-(C₁₋₆ alkylamino)-C₁₋₆ alkyl, amino, C₁₋₆ alkylamino, C₃₋₇ cycloalkylamino, di-(C₁₋₆ alkyl)amino, amino-C₁₋₆ alkylamino, C₁₋₆ alkoxy-C₁₋₆ alkylamino, C₁₋₆ alkoxycarbonyl-C₁₋₆ alkylamino, di-(C₁₋₆ alkoxy-C₁₋₆ alkyl)amino, aminocarbonyl, C₁₋₆ alkylaminocarbonyl, di-(C₁₋₆ alkyl)aminocarbonyl, C₃₋₇ cycloalkylaminocarbonyl, C₁₋₆ alkoxy, C₃₋₇ cycloalkoxy, hydroxyl-C₁₋₆ alkoxy, C₁₋₆ haloalkoxy, amino-C₁₋₆ alkyl, amino-C₁₋₆ alkoxy, C₁₋₆ alkylsulfonyl, C₂₋₆ alkenylsulfonyl, C₃₋₇ cycloalkylsulfonyl, heterocyclyl optionally substituted by B, aryl optionally substituted by B, heteroaryl optionally substituted by B, C₁₋₆ alkylsulfonylamino, C₂₋₆ alkenylsulfonylamino, C₃₋₇ cyclooalkylsulfonylamino, amido, C₁₋₆ alkylcarbonylamino, C₂₋₆ alkenylcarbonylamino, C₃₋₇ cyclooalkylcarbonylamino, C₁₋₆ alkoxycarbonylamino, C₃₋₇ cycloalkoxycarbonylamino, ureido, C₃₋₇ cycloalkyl, C₃₋₇ halocycloalkyl, heterocyclyl-oxy, piperidinylamino, N-methyl-piperidinyl-4-carbonyl, piperazinyl-C₁₋₆ alkyl, pyrrolylcarbonylamino, N-methyl-piperidinylcarbonylamino or heterocyclyl-C₁₋₆ alkoxy; or
R₃ and R₄ together form a 3 to 8-membered ring with the atoms in the aromatic ring to which they are attached; and
B is hydrogen, C₁₋₆ alkyl, C₁₋₆ haloalkyl, halo, hydroxyl, aryl, amino, C₁₋₆ alkylamino, C₃₋₇ cycloalkylamino, di-(C₁₋₆ alkyl)amino, cyano, or C₃₋₇ cycloalkyl.

In one embodiment, the tyrosine kinase inhibitor is CBT-102, which has the following structures:

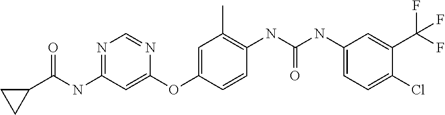 [SMILES: Cc1cc(Oc2cc(NC(=O)C3CC3)ncn2)ccc1NC(=O)Nc1ccc(Cl)c(C(F)(F)F)c1]

In certain embodiments, the expression level is an RNA level, a protein level, or a protein activation level. In certain embodiments, the expression level of the protein kinase is measured by an amplification assay, a hybridization assay, a sequencing assay or an array, or an antibody-based assay such as western-blot, immunohistochemistry (IHC) or ELISA. In certain embodiments, the protein activation level of the kinase is measured by detecting the phosphorylation of the protein kinase.

In certain embodiments, the cancer is selected from the groups consisting of gastric cancer, a lung cancer, esophageal cancer, a melanoma, a renal cancer, a liver cancer, a myeloma, a prostate cancer, a breast cancer, a colorectal cancer, a pancreatic cancer, a thyroid cancer, a hematological cancer, a leukemia and a non-Hodgkin's lymphoma. In some embodiment, the cancer is gastric cancer, lung cancer, colorectal cancer, liver cancer, esophageal cancer, renal cancer or breast cancer.

In another aspect, the present disclosure provides a method for continuing a cancer therapy in a patient. In one embodiment, the method comprises: a) treating the patient with a tyrosine kinase inhibitor; b) obtaining a tumor sample from the patient; c) measuring the expression level of a first protein kinase; d) comparing the expression level of the first protein kinase to a corresponding reference expression level; e) determining a likelihood of the patient being responsive to the tyrosine kinase inhibitor; and f) continuing treatment of the cancer when the expression level of the first protein kinase in the tumor sample demonstrates responsiveness.

In still another aspect, the present disclosure provides use of an agent of measuring the expression level of a first protein kinase in manufacture of a kit for determining the likelihood of a patient being responsive to a cancer treatment using a tyrosine kinase inhibitor. In one embodiment, the first protein kinase is selected from the group consisting of DDR1, CSF1R, CDKL2, cKit, c-RAF, Flt1, Flt4, KDR, MAP4K5, PDGFRα, PTK5, Ret, SAPK2b and ZAK. In certain embodiments, the agent is a primer or an antibody.

In yet another aspect, the present disclosure provides a kit for determining the likelihood of a patient being responsive to a cancer treatment using a tyrosine kinase inhibitor. In certain embodiments, the kit comprises an agent of measuring the expression level of a first protein kinase in a sample obtained from the patient. In certain embodiments, the agent is a primer or an antibody. In certain embodiments, the first protein kinase is selected from the group consisting of DDR1, CSF1R, CDKL2, cKit, c-RAF, Flt1, Flt4, KDR, MAP4K5, PDGFRα, PTK5, Ret, SAPK2b and ZAK. In one embodiment, the kit further comprises a secondary antibody.

### BRIEF DESCRIPTION OF DRAWING

FIG. 1 illustrates the in vivo efficacy of CBT-102 in a group of PDX models.

FIG. 2A illustrates CBT-102 efficacy vs. DDR1 expression in the PDX tumor; FIG. 2B illustrates the statistical results about the relationship between DDR1 expression and the efficacy of CBT-102 in PDX tumors.

FIG. 3 A illustrates CBT-102 efficacy vs. DDR1 expression in lung cancer;

FIG. 3B illustrates the statistical results about the relationship between DDR1 expression and the efficacy of CBT-102 in lung cancer.

FIG. 4A illustrates the IC50 determination on M-NFS-60 cells; FIG. 4B illustrates the IC50 determination on RAW264.7 cells.

FIG. 5 illustrates the CBT-102 efficacy in MC38 modal.

FIG. 6 illustrates that CBT-102 reduced F4-80 IHC score in MC-38 tumor.

FIG. 7 illustrates that CBT-102 had no significant effect on T cell surface markers CD3, CD4, and CD8 but on macrophage surface marker F4-80 in MC-38 tumors.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

The term “amount” or “level” refers to the quantity of a polynucleotide of interest or a polypeptide of interest present in a sample. Such quantity may be expressed in the absolute terms, i.e., the total quantity of the polynucleotide or polypeptide in the sample, or in the relative terms, i.e., the concentration of the polynucleotide or polypeptide in the sample.

As used herein, the term “administering” means providing a pharmaceutical agent or composition to a subject, and includes, but is not limited to, administering by a medical professional and self-administering.

As used herein, an “antibody” encompasses naturally occurring immunoglobulins as well as non-naturally occurring immunoglobulins, including, for example, single chain antibodies, chimeric antibodies (e.g., humanized murine antibodies), and heteroconjugate antibodies (e.g., bispecific antibodies). Fragments of antibodies include those that bind antigen, (e.g., Fab′, F(ab′)2, Fab, Fv, and rIgG). See also, e.g., Pierce Catalog and Handbook, 1994-1995 (Pierce Chemical Co., Rockford, Ill.); Kuby, I, Immunology, 3rd Ed., W.H. Freeman & Co., New York (1998). The term antibody also includes bivalent or bispecific molecules, diabodies, triabodies, and tetrabodies. The term “antibody” further includes both polyclonal and monoclonal antibodies.

As used herein, the term “cancer” refers to any diseases involving an abnormal cell growth and includes all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells. As used herein, cancer types include, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, breast cancer, Burkitt's lymphoma, cerebellar astrocytoma, cerebral astrocytoma/malignant glioma, cervical cancer, chronic lymphocytic leukemia, chronic myelogenous leukemia, colon cancer, emphysema, endometrial cancer, ependymoma, esophageal cancer, Ewing family of tumors, Ewing's sarcoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukaemia, liver cancer, lung cancer, medulloblastoma, melanoma, neuroblastoma, non-Hodgkin lymphoma, ovarian cancer, pancreatic cancer, pharyngeal cancer, prostate cancer, rectal cancer, renal cell carcinoma (kidney cancer), retinoblastoma, skin cancer, stomach cancer, supratentorial primitive neuroectodermal tumors, testicular cancer, throat cancer, thyroid cancer, vaginal cancer, visual pathway and hypothalamic glioma.

The term “cancer sample” includes a biological sample or a sample from a biological source that contains one or more cancer cells. Biological samples include samples from body fluids, e.g., blood, plasma, serum, or urine, or samples derived, e.g., by biopsy, from cells, tissues or organs, preferably tumor tissue suspected to include or essentially consist of cancer cells.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ, e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell; a cell from an endocrine system or organ, e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte); a cell from a nervous system or organ, e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph); a cell from a respiratory system or organ, e.g., a pneumocyte (a type I pneumocyte and a type II pneumocyte), a clara cell, a goblet cell, an alveolar macrophage; a cell from circular system or organ, e.g., myocardiocyte and pericyte; a cell from digestive system or organ, e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell); a cell from integumentary system or organ, e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell), a muscle cell (e.g., myocyte), an adipocyte, a fibroblast, and a tendon cell), a cell from urinary system or organ (e.g., a podocyte, a juxtaglomerular cell, an intragiomerular mesangial cell, an extragiomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell).

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%>, 70%>, 80%>, 90%, and 100% complementary).

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

The terms “determining,” “assessing,” “assaying,” “measuring” and “detecting” can be used interchangeably and refer to both quantitative and semi-quantitative determinations. Where either a quantitative and semi-quantitative determination is intended, the phrase “determining a level” of a polynucleotide or polypeptide of interest or “detecting” a polynucleotide or polypeptide of interest can be used.

The term “hybridizing” refers to the binding, duplexing, or hybridizing of a nucleic acid molecule preferentially to a particular nucleotide sequence under stringent conditions. The term “stringent conditions” refers to hybridization and wash conditions under which a probe will hybridize preferentially to its target subsequence, and to a lesser extent to, or not at all to, other sequences in a mixed population (e.g., a cell lysate or DNA preparation from a tissue biopsy). A stringent condition in the context of nucleic acid hybridization are sequence dependent, and are different under different environmental parameters. An extensive guide to the hybridization of nucleic acids is found in, e.g., Tijssen Laboratory Techniques in Biochemistry and Molecular Biology—Hybridization with Nucleic Acid Probes part I, Ch. 2, “Overview of principles of hybridization and the strategy of nucleic acid probe assays,” (1993) Elsevier, N.Y. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and Russell Molecular Cloning; A Laboratory Manual (3rd ed.) Vol. 1-3 (2001) Cold Spring Harbor Laboratory, Cold Spring Harbor Press, NY). An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4×SSC to 6×SSC at 40° C. for 15 minutes.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The term “Patient derived xenografts (PDX)” refer to models of cancer where the tissue or cells from a patient's tumor are implanted into an immunodeficient or humanized mouse. PDX models are used to create an environment that allows for the natural growth of cancer, its monitoring, and corresponding treatment evaluations for the original patient.

“Primer” as used herein refers to an oligonucleotide molecule with a length of 7-40 nucleotides, preferably 10-38 nucleotides, preferably 15-30 nucleotides, or 15-25 nucleotides, or 17-20 nucleotides. For example, the primer can an oligonucleotide having a length of 7, 8, 9, 10, 11, 12, 13, 14, 15, 16, 17, 18, 19, 20, 21, 22, 23, 24, 25, 26, 27, 28, 29 or 30 nucleotides. Primers are usually used in the amplification of a DNA sequence by polymerase chain reaction (PCR) as well known in the art. For a DNA template sequence to be amplified, a pair of primers can be designed at its 5′ upstream and its 3′ downstream sequence, i.e. 5′ primer and 3′ primer, each of which can specifically hybridize to a separate strand of the DNA double strand template. 5′ primer is complementary to the anti-sense strand of the DNA double strand template; and 3′ primer is complementary to the sense strand of the DNA template. As known in the art, the “sense strand” of a double stranded DNA template is the strand which contains the sequence identical to the mRNA sequence transcribed from the DNA template (except that “U” in RNA corresponds to “T” in the DNA) and encoding for a protein product. The complementary sequence of the sense strand is the “anti-sense strand.” In the present disclosure, all the SEQ ID NOs are sense strand DNA, and the sequences to which the SEQ ID NOs are complementary are anti-sense strand DNA.

The terms “responsive,” “clinical response,” “positive clinical response,” and the like, as used in the context of a patient's response to a cancer therapy, are used interchangeably and refer to a favorable patient response to a treatment as opposed to unfavorable responses, i.e. adverse events. In a patient, beneficial response can be expressed in terms of a number of clinical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment. In a population the clinical benefit of a drug, i.e., its efficacy can be evaluated on the basis of one or more endpoints. For example, analysis of overall response rate (ORR) classifies as responders those patients who experience CR or PR after treatment with drug. Analysis of disease control (DC) classifies as responders those patients who experience CR, PR or SD after treatment with drug. A positive clinical response can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition of metastasis; (6) enhancement of anti-turn or immune response, possibly resulting in regression or rejection of the tumor; (7) relief, to some extent, of one or more symptoms associated with the tumor; (8) increase in the length of survival following treatment; and/or (9) decreased mortality at a given point of time following treatment. Positive clinical response may also be expressed in terms of various measures of clinical outcome. Positive clinical outcome can also be considered in the context of an individual's outcome relative to an outcome of a population of patients having a comparable clinical diagnosis, and can be assessed using various endpoints such as an increase in the duration of recurrence-free interval (RFI), an increase in the time of survival as compared to overall survival (OS) in a population, an increase in the time of disease-free survival (DFS), an increase in the duration of distant recurrence-free interval (DRFI), and the like. Additional endpoints include a likelihood of any event (AE)-free survival, a likelihood of metastatic relapse (MR)-free survival (MRFS), a likelihood of disease-free survival (DFS), a likelihood of relapse-free survival (RFS), a likelihood of first progression (FP), and a likelihood of distant metastasis-free survival (DMFS). An increase in the likelihood of positive clinical response corresponds to a decrease in the likelihood of cancer recurrence or relapse.

The term “standard control” or “reference level” as used herein refers to a predetermined amount or concentration of a polynucleotide sequence or polypeptide sequence that is present in an established reference, e.g., a normal tissue sample, a healthy, non-cancer tissue sample, or a diploid, non-transformed, non-cancerous, genomically stable healthy human cell line. The standard control or reference value is suitable for the use of a method of the present invention, to serve as a basis for comparing the amount of a specific mRNA or protein that is present in a test sample. An established sample serving as a standard control provides an average amount of a specific mRNA or protein that is typical in a normal tissue sample. A standard control value may vary depending on the nature of the sample as well as other factors such as the gender, age, ethnicity of the subjects based on whom such a control value is established.

The term “sample” as used herein refers to a biological sample that is obtained from a subject of interest. Examples of sample include, without limitation, bodily fluid, such as blood, plasma, serum, urine, vaginal fluid, uterine or vaginal flushing fluids, plural fluid, ascitic fluid, cerebrospinal fluid, saliva, sweat, tears, sputum, bronchioalveolar lavage fluid, etc., and tissues, such as biopsy tissue (e.g. biopsied bone tissue, bone marrow, breast tissue, gastrointestinal tract tissue, lung tissue, liver tissue, prostate tissue, brain tissue, nerve tissue, meningeal tissue, renal tissue, endometrial tissue, cervical dittuse, lymph node tissue, muscle tissue, or skin tissue), a paraffin embedded tissue. In certain embodiments, the sample can be a biological sample comprising cancer cells. In some embodiments, the sample is a fresh or archived sample obtained from a tumor, e.g., by a tumor biopsy or fine needle aspirate. The sample also can be any biological fluid containing cancer cells. The collection of a sample from a subject is performed in accordance with the standard protocol generally followed by hospital or clinics, such as during a biopsy.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “treatment,” “treat,” or “treating” refer to a method of reducing the effects of a cancer (e.g., breast cancer, lung cancer, ovarian cancer or the like) or symptom of cancer. Thus, in the disclosed method, treatment can refer to a 10%, 20%, 30%, 40%, 50%, 60%, 70%), 80%), 90%), or 100% reduction in the severity of an cancer or symptom of the cancer. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

The term “tumor sample” includes a biological sample or a sample from a biological source that contains one or more tumor cells. Biological samples include samples from body fluids, e.g., blood, plasma, serum, or urine, or samples derived, e.g., by biopsy, from cells, tissues or organs, preferably tumor tissue suspected to include or essentially consist of cancer cells.

Protein Kinase Expression Level

The methods and compositions described herein are based, in part, on the discovery of protein kinases whose expression level in cancer samples is indicative of responsiveness of cancer patients to a tyrosine kinase inhibitor.

Protein kinases are a group of enzymes that modify other proteins by chemically adding phosphate groups to the target proteins, i.e., phosphorylation. Phosphorylation usually results in a functional change of the target protein by changing enzyme activity, cellular location, or association with other proteins. Phosphorylation of proteins by kinases is an important mechanism in communicating signals within a cell (signal transduction) and regulating cellular activity, such as cell division. As a result, protein kinases function as an “on” or “off” switch in many cellular functions. Protein kinases can become mutated, stuck in the “on” position, and cause unregulated growth of the cell, which is a necessary step for the development of cancer. Therefore, kinase inhibitors, such as imatinib, are often effective cancer treatments. The human genome contains about 500 protein kinase genes. Most protein kinases can be categorized into subclasses: serine/threonine kinases and tyrosine kinases.

Serine/threonine kinases phosphorylate the OH group of serine or threonine residues in a target protein. Examples of serine/threonine kinases include MAP kinases, ERK family, the stress-activated protein kinase JNK and p38.

Tyrosine kinases, a subclass of protein kinase, are a group of enzymes that can transfer a phosphate group from ATP to a protein in a cell, in which the phosphate group is attached to the amino acid residue tyrosine on the protein. Most tyrosine kinases have an associated protein tyrosine phosphatase, which removes the phosphate group.

In certain embodiments, the kinase is selected from the group consisting of DDR1, CSF1R, CDKL2, cKit, c-RAF, Flt1, Flt4, KDR, MAP4K5, PDGFRα, PTK5, Ret, SAPK2b and ZAK. In certain embodiments, the kinase contains a mutation. In one embodiment, the mutation is cKit(V560G) or PDGFRα (V561D). In one embodiment, the protein kinase is DDR1 or CSF1R.

DDR1 as used herein refers to the human gene Discoidin domain receptor family, member 1, also known as CD167a (cluster of differentiation 167a). DDR1 encodes a tyrosine kinase that is widely expressed in normal and transformed epithelial cells and is activated by various types of collagen. DDR1 protein belongs to a subfamily of tyrosine kinase receptors with a homology region to the Dictyostelium discoideum protein discoidin I in their extracellular domain. Its autophosphorylation is achieved by all collagens so far tested (type I to type VI). A closely related family member is the DDR2 protein (Fu H L et al. (March 2013) The Journal of Biological Chemistry 288 (11): 7430-7). In situ studies and Northern-blot analysis showed that expression of DDR is restricted to epithelial cells, particularly in the kidney, lung, gastrointestinal tract, and brain. In addition, DDR1 is significantly over-expressed in several human tumors from breast, ovarian, esophageal, and pediatric brain. DDR1 gene is located on chromosome 6p21.3 in proximity to several HLA class I genes. Alternative splicing of this gene results in multiple transcript variants (“Entrez Gene: DDR1 discoidin domain receptor family, member 1”). In certain embodiments, the DDR1 mRNA has the sequence of SEQ ID NO: 1 and the DDR1 protein has the sequence of SEQ ID NO: 2.

CSF1R as used herein refers to Colony stimulating factor 1 receptor, also known as macrophage colony-stimulating factor receptor (M-CSFR), and CD115 (Cluster of Differentiation 115), which is a cell-surface protein encoded, in humans, by the CSF1R gene (known also as c-FMS) (EntrezGene 1436; Galland F, Stefanova M, Lafage M, Birnbaum D (1992) Cell Genet 60 (2): 114-6). The CSF1R protein has 972 amino acids and a predicted molecular weight of 108 kD. The CSF1R protein is a single pass type I membrane protein and acts as the receptor for colony stimulating factor 1, a cytokine which controls the production, differentiation, and function of macrophages. CSF1R mediates most, if not all, of the biological effects of CSF1. The CSF1R is a tyrosine kinase transmembrane receptor and member of the CSF1/PDGF receptor family of tyrosine-protein kinase (Xu Q et al. (2015) Science Signaling. 8 (405): rs13; Meyers M J et al. (2010) Bioorganic & Medicinal Chemistry Letters. 20 (5): 1543-7.) Ligand binding activates CSF1R through a process of oligomerization and trans-phosphorylation. Because CSF1R is overexpressed in many cancers and on tumor-associated macrophages (TAM), CSF1R inhibitors (and CSF1 inhibitors) have been studied for many years as a possible treatment for cancer or inflammatory diseases (Patel S, Player M R (2009) Curr Top Med Chem. 9 (7): 599-610; Cannarile M A et al. (2017) Journal for Immunotherapy of Cancer. 5 (1): 53). In certain embodiments, the CSF1R mRNA has the sequence of SEQ ID NO: 3 and the CSF1R protein has the sequence of SEQ ID NO: 4.

CDKL2 as used herein refers to Cyclin-dependent kinase-like 2, which is an enzyme that in humans is encoded by the CDKL2 gene (Taglienti C A, Wysk M, Davis R J (February 1997) Oncogene. 13 (12): 2563-74; “Entrez Gene: CDKL2 cyclin-dependent kinase-like 2 (CDCl₂-related kinase)”). CDKL2 is a member of a large family of CDCl₂-related serine/threonine protein kinases. It accumulates primarily in the cytoplasm, with lower levels in the nucleus (“Entrez Gene: CDKL2 cyclin-dependent kinase-like 2 (CDCl₂-related kinase)”).

cKit as used herein refers to proto-oncogene c-KIT, also known as tyrosine-protein kinase KIT, CD117 (cluster of differentiation 117) or mast/stem cell growth factor receptor (SCFR), which is a receptor tyrosine kinase protein that in humans is encoded by the KIT gene (Andre C et al. (January 1997) Genomics. 39 (2): 216-26). Multiple transcript variants encoding different isoforms have been found for this gene (“Entrez Gene: KIT v-kit Hardy-Zuckerman 4 feline sarcoma viral oncogene homolog”; National Cancer Institute Dictionary of Cancer Terms, c-kit. Accessed Oct. 13, 2014.). KIT was first described by the German biochemist Axel Ullrich in 1987 as the cellular homolog of the feline sarcoma viral oncogene v-kit (Yarden Y et al. (November 1987) EMBO J. 6 (11): 3341-51).

cKit is a cytokine receptor expressed on the surface of hematopoietic stem cells as well as other cell types. Altered forms of this receptor may be associated with some types of cancer (Edling C E, Hallberg B (2007) Int. J. Biochem. Cell Biol. 39 (11): 1995-8). cKit is a receptor tyrosine kinase type III, which binds to stem cell factor (a substance that causes certain types of cells to grow), also known as “steel factor” or “c-kit ligand”. When cKit receptor binds to stem cell factor (SCF), it forms a dimer that activates its intrinsic tyrosine kinase activity, that in turn phosphorylates and activates signal transduction molecules that propagate the signal in the cell (Blume-Jensen P et al. (1991 Dec. 10) EMBO Journal. 10 (13): 4121-4128). After activation, the receptor is ubiquitinated to mark it for transport to a lysosome and eventual destruction. Signaling through cKit plays a role in cell survival, proliferation, and differentiation. For instance, cKit signaling is required for melanocyte survival, and it is also involved in haematopoiesis and gametogenesis (Brooks, Samantha (2006), Studies of genetic variability at the KIT locus and white spotting patterns in the horse (Thesis), University of Kentucky Doctoral Dissertations, pp. 13-16). Activating mutations in cKit gene are associated with gastrointestinal stromal tumors, testicular seminoma, mast cell disease, melanoma, acute myeloid leukemia, while inactivating mutations are associated with the genetic defect piebaldism (“Entrez Gene: KIT v-kit Hardy-Zuckerman 4 feline sarcoma viral oncogene homolog”).

c-RAF as used herein refers to RAF proto-oncogene serine/threonine-protein kinase, also known as proto-oncogene c-RAF or simply Raf-1, which is an enzyme (Li P et al. (1991) Cell. 64 (3): 479-82.) that in humans is encoded by the RAF1 gene (Rapp U R et al. (1983). Proc. Natl. Acad. Sci. U.S.A. 80 (14): 4218-22; Bonner T et al. (1984) Science 223 (4631): 71-4). The c-Raf protein is part of the ERK1/2 pathway as a MAP kinase (MAP3K) that functions downstream of the Ras subfamily of membrane associated GTPases (“Entrez Gene: RAF1 v-raf-1 murine leukemia viral oncogene homolog 1”).

Flt1 as used herein refers to vascular endothelial growth factor receptor 1 (VEGFR1), which is a protein that in humans is encoded by the FLT1 gene (Shibuya M et al. (April 1990) Oncogene 5 (4): 519-24). Flt1 belongs to the src gene family and is related to oncogene ROS (MIM 165020). Like other members of this family, Flt1 shows tyrosine protein kinase activity that is important for the control of cell proliferation and differentiation. The ablation of Flt1 by chemical and genetic means has recently been found to augment the conversion of white adipose tissue to brown adipose tissue as well as increase brown adipose angiogenesis in mice (Seki T et al. (2018) The Journal of Experimental Medicine. 215 (2): 611-626). Functional genetic variation in FLT1 (rs9582036) has been found to affect non-small cell lung cancer survival (Glubb D M et al. (2015) Journal of Thoracic Oncology. 10 (7): 1067-75.).

Flt4 as used herein refers to Fms-related tyrosine kinase 4, which is a protein that in humans is encoded by the FLT4 gene (“Entrez Gene: FLT4 fms-related tyrosine kinase 4”; Galland F et al. (1992) Genomics 13 (2): 475-8.). Flt4 gene encodes a tyrosine kinase receptor for vascular endothelial growth factors C and D. The Flt4 protein is thought to be involved in lymphangiogenesis and maintenance of the lymphatic endothelium. Mutations in Flt4 gene cause hereditary lymphedema type IA (“Entrez Gene: FLT4 fms-related tyrosine kinase 4”).

KDR as used herein refers to kinase insert domain receptor (KDR, a type IV receptor tyrosine kinase) also known as vascular endothelial growth factor receptor 2 (VEGFR-2), CD309 (cluster of differentiation 309), and Flk1 (Fetal Liver Kinase 1). The Q472H germline KDR genetic variant affects VEGFR-2 phosphorylation and has been found to associate with microvessel density in NSCLC (Glubb D M et al. (August 2011) Clinical Cancer Research. 17 (16): 5257-67). KDR has been shown to interact with SHC2, Annexin A5 and SHC1 (Warner A J et al. (April 2000) The Biochemical Journal 347 (Pt 2): 501-9; Wen Y et al. (May 1999) Biochemical and Biophysical Research Communications. 258 (3): 713-21; Zanetti A et al. (April 2002) Arteriosclerosis, Thrombosis, and Vascular Biology 22 (4): 617-22; D'Angelo G et al. (May 1999) Molecular Endocrinology 13 (5): 692-704).

MAP4K as used herein refers to mitogen-activated protein kinase kinase kinase kinase 5, which is an enzyme that in humans is encoded by the MAP4K5 gene (Tung R M, Blenis J (1997) Oncogene 14 (6): 653-9; Schultz S J, Nigg E A (1994) Cell Growth Differ 4 (10): 821-30; “Entrez Gene: MAP4K5 mitogen-activated protein kinase kinase kinase kinase 5”). MAP4K is a member of the serine/threonine protein kinase family, that is highly similar to yeast SPS1/STE20 kinase. Yeast SPS1/STE20 functions near the beginning of the MAP kinase signal cascades that is essential for yeast pheromone response. MAP4K was shown to activate Jun kinase in mammalian cells, which suggested a role in stress response. Two alternatively spliced transcript variants encoding the same protein have been described for this gene (“Entrez Gene: MAP4K5 mitogen-activated protein kinase kinase kinase kinase 5”). MAP4K5 has been shown to interact with CRKL and TRAF2 (Shi, C S; Tuscano J; Kehrl J H (2000) Blood 95 (3): 776-82; Shi, C S et al. (1999) J. Immunol. 163 (6): 3279-85).

PDGFRα as used herein refers to platelet-derived growth factor receptor α, which is a receptor located on the surface of a wide range of cell types. PDGFRα binds to certain isoforms of platelet-derived growth factors (PDGFs) and thereby becomes active in stimulating cell signaling pathways that elicit responses such as cellular growth and differentiation. PDGFRα is critical for the development of certain tissues and organs during embryogenesis and for the maintenance of these tissues and organs, particularly hematologic tissues, throughout life. Mutations in the gene which codes for PDGFRα, i.e. the PDGFRa gene, are associated with an array of clinically significant neoplasms. The molecular mass of the mature, glycosylated PDGFRα protein is approximately 170 kD.

PTK5 as used herein refers to tyrosine protein kinase 5 also known as fyn-related kinase (FRK), which is encoded by the FRK gene (Lee J et al. (April 1994) Gene. 138 (1-2): 247-51; “Entrez Gene: FRK fyn-related kinase”). The protein encoded by this gene belongs to the tyrosine kinase family of protein kinases. This tyrosine kinase is a nuclear protein and may function during G1 and S phase of the cell cycle and suppress growth (“Entrez Gene: FRK fyn-related kinase”). FRK has been shown to interact with retinoblastoma protein (R J Craven, W G Cance, E T Liu (1995) Cancer Res 55 (18): 3969-72).

Ret as used herein refers to the RET proto-oncogene which encodes a receptor tyrosine kinase for members of the glial cell line-derived neurotrophic factor (GDNF) family of extracellular signaling molecules (Knowles P P, Murray-Rust J, Kjaer S, et al. (2006) J. Biol. Chem. 281 (44): 33577-87). RET loss of function mutations are associated with the development of Hirschsprung's disease, while gain of function mutations are associated with the development of various types of human cancer, including medullary thyroid carcinoma, multiple endocrine neoplasias type 2A and 2B, pheochromocytoma and parathyroid hyperplasia.

SAPK2b also known as “MAPK11” refers to mitogen-activated protein kinase 11, which is an enzyme that in humans is encoded by the MAPK11 gene (Goedert M et al. (August 1997) EMBO J. 16 (12): 3563-71; “Entrez Gene: MAPK11 mitogen-activated protein kinase 11”). SAPK2b protein is a member of the MAP kinase family. MAP kinases act as an integration point for multiple biochemical signals, and are involved in a wide variety of cellular processes such as proliferation, differentiation, transcription regulation, and development. SAPK2b is most closely related to p38 MAP kinase, both of which can be activated by proinflammatory cytokines and environmental stress. This kinase is activated through its phosphorylation by MAP kinase kinases (MKKs), preferably by MKK6.

Transcription factor ATF2/CREB2 has been shown to be a substrate of this kinase. (“Entrez Gene: MAPK11 mitogen-activated protein kinase 11”) MAPK11 has been shown to interact with HDAC3 and Promyelocytic leukemia protein (Mahlknecht U et al. (2004) J. Immunol. 173 (6): 3979-90; Shin J et al. (2004) J. Biol. Chem. 279 (39): 40994-1003).

ZAK as used herein refers to sterile alpha motif and leucine zipper containing kinase AZK, which is a member of the MAPKKK family of signal transduction molecules. ZAK protein has an N-terminal kinase catalytic domain, followed by a leucine zipper motif and a sterile-alpha motif (SAM). This magnesium-binding protein forms homodimers and is located in the cytoplasm. The ZAK protein mediates gamma radiation signaling leading to cell cycle arrest and activity of this protein plays a role in cell cycle checkpoint regulation in cells. The ZAK protein also has pro-apoptotic activity. Alternate transcriptional splice variants, encoding different isoforms, have been characterized (“Entrez Gene: ZAK sterile alpha motif and leucine zipper containing kinase AZK”; Liu et al. (2000) Biochemical and Biophysical Research Communications 274 (3): 811-816.). ZAK has been shown to interact with ZNF33A (Yang, Jaw-Ji (January 2003) Biochem. Biophys. Res. Commun. 301 (1): 71-7).

Detection Reagents for Kinase Expression

In one aspect, the present disclosure provides detection reagents for detecting the kinase expression disclosed herein.

In certain embodiments, the detection reagents comprise primers or probes that can hybridize to the polynucleotide of the protein kinase gene or protein kinase mRNA.

The term “primer” as used herein refers to oligonucleotides that can specifically hybridize to a target polynucleotide sequence, due to the sequence complementarity of at least part of the primer within a sequence of the target polynucleotide sequence. A primer can have a length of at least 8 nucleotides, typically 8 to 70 nucleotides, usually of 18 to 26 nucleotides. For proper hybridization to the target sequence, a primer can have at least 75%, at least 80%, at least 85%, at least 90%, or at least 95% sequence complementarity to the hybridized portion of the target polynucleotide sequence.

Oligonucleotides useful as primers may be chemically synthesized according to the solid phase phosphoramidite triester method first described by Beaucage and Caruthers, Tetrahedron Letts. (1981) 22: 1859-1862, using an automated synthesizer, as described in Needham-Van Devanter et al, Nucleic Acids Res. (1984) 12:6159-6168.

Primers are useful in nucleic acid amplification reactions in which the primer is extended to produce a new strand of the polynucleotide. Primers can be readily designed by a skilled artisan using common knowledge known in the art, such that they can specifically anneal to the nucleotide sequence of the target nucleotide sequence of the protein kinase gene provided herein. Usually, the 3′ nucleotide of the primer is designed to be complementary to the target sequence at the corresponding nucleotide position, to provide optimal primer extension by a polymerase.

The term “probe” as used herein refers to oligonucleotides or analogs thereof that can specifically hybridize to a target polynucleotide sequence, due to the sequence complementarity of at least part of the probe within a sequence of the target polynucleotide sequence. Exemplary probes can be, for example DNA probes, RNA probes, or protein nucleic acid (PNA) probes. A probe can have a length of at least 8 nucleotides, typically 8 to 70 nucleotides, usually of 18 to 26 nucleotides. For proper hybridization to the target sequence, a probe can have at least 75%, at least 80%, at least 85%, at least 90%, or at least 95% sequence complementarity to hybridized portion of the target polynucleotide sequence. Probes and also be chemically synthesized according to the solid phase phosphoramidite triester method as described above. Methods for preparation of DNA and RNA probes, and the conditions for hybridization thereof to target nucleotide sequences, are described in Molecular Cloning: A Laboratory Manual, J. Sambrook et al., eds., 2nd edition. Cold Spring Harbor Laboratory Press, 1989, Chapters 10 and 11.

In certain embodiments, the primers and the probes provided herein are detectably labeled. Examples of the detectable label suitable for labeling primers and probes include, for example, chromophores, radioisotopes, fluorophores, chemiluminescent moieties, particles (visible or fluorescent), nucleic acids, ligand, or catalysts such as enzymes.

In certain embodiments, the detection reagents comprise an antibody that specifically binds to the protein kinase protein.

The term “antibody” as used herein refers to an immunoglobulin or an antigen-binding fragment thereof, which can specifically bind to a target protein antigen. Antibodies can be identified and prepared by selection of antibodies from libraries of recombinant antibodies in phage or similar vectors, as well as preparation of polyclonal and monoclonal antibodies by immunizing animals such as rabbits or mice (see, e.g., Huse et al., Science (1989) 246:1275-1281; Ward et al, Nature (1989) 341:544-546).

It can be understood that in certain embodiments, the antibodies are modified or labeled to be properly used in various detection assays. In certain embodiments, the antibody is detectably labeled.

Sample Preparation

Any biological sample suitable for conducting the methods provided herein can be obtained from the subject. In certain embodiments, the sample can be further processed by a desirable method for performing the detection of the protein kinase expression.

In certain embodiments, the method further comprises isolating or extracting cancer cell (such as circulating tumor cell) from the biological fluid sample (such as peripheral blood sample) or the tissue sample obtained from the subject. The cancer cells can be separated by immunomagnetic separation technology such as that available from Immunicon (Huntingdon Valley, Pa.).

In certain embodiments, a tissue sample can be processed to perform in situ hybridization. For example, the tissue sample can be paraffin-embedded before fixing on a glass microscope slide, and then deparaffinized with a solvent, typically xylene.

In certain embodiments, the method further comprises isolating the nucleic acid, e.g. DNA or RNA from the sample. Various methods of extraction are suitable for isolating the DNA or RNA from cells or tissues, such as phenol and chloroform extraction, and various other methods as described in, for example, Ausubel et al., Current Protocols of Molecular Biology (1997) John Wiley & Sons, and Sambrook and Russell, Molecular Cloning; A Laboratory Manual 3^{rd} ed (2001).

Commercially available kits can also be used to isolate DNA and/or RNA, including for example, the NucliSens extraction kit (Biomerieux, Marcy l'Etoile, France), QIAamp™ mini blood kit, Agencourt Genfind™, Rneasy® mini columns (Qiagen), PureLink® RNA mini kit (Thermo Fisher Scientific), and Eppendorf Phase Lock Gels™. A skilled person can readily extract or isolate RNA or DNA following the manufacturer's protocol.

Methods of Detecting Protein Kinase Expression Level

The methods of the present disclosure include detecting the protein kinase expression level described herein in a sample obtained from a subject having cancer or suspected of having cancer. The protein kinase expression level can be detected in the RNA (e.g. mRNA) level, protein level or protein activation level using proper methods known in the art including, without limitation, an amplification assay, a hybridization assay, a sequencing assay, and an immunoassay.

Amplification Assay

A nucleic acid amplification assay involves copying a target nucleic acid (e.g. DNA or RNA), thereby increasing the number of copies of the amplified nucleic acid sequence. Amplification may be exponential or linear. Exemplary nucleic acid amplification methods include, but are not limited to, amplification using the polymerase chain reaction (“PCR”, see U.S. Pat. Nos. 4,683,195 and 4,683,202; PCR Protocols: A Guide To Methods And Applications (Innis et al., eds, 1990)), reverse transcriptase polymerase chain reaction (RT-PCR), quantitative real-time PCR (qRT-PCR); quantitative PCR, such as TaqMan®, nested PCR, ligase chain reaction (See Abravaya, K., et al., Nucleic Acids Research, 23:675-682, (1995), branched DNA signal amplification (see, Urdea, M. S., et al., AIDS, 7 (suppl 2):S11-S14, (1993), amplifiable RNA reporters, Q-beta replication (see Lizardi et al., Biotechnology (1988) 6: 1197), transcription-based amplification (see, Kwoh et al., Proc. Natl. Acad. Sci. USA (1989) 86: 1173-1177), boomerang DNA amplification, strand displacement activation, cycling probe technology, self-sustained sequence replication (Guatelli et al., Proc. Natl. Acad. Sci. USA (1990) 87:1874-1878), rolling circle replication (U.S. Pat. No. 5,854,033), isothermal nucleic acid sequence based amplification (NASBA), and serial analysis of gene expression (SAGE).

In certain embodiments, the nucleic acid amplification assay is a PCR-based method. PCR is initiated with a pair of primers that hybridize to the target nucleic acid sequence to be amplified, followed by elongation of the primer by polymerase which synthesizes the new strand using the target nucleic acid sequence as a template and dNTPs as building blocks. Then the new strand and the target strand are denatured to allow primers to bind for the next cycle of extension and synthesis. After multiple amplification cycles, the total number of copies of the target nucleic acid sequence can increase exponentially.

In certain embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signal as a function of temperature because melting point of template sequences will generally be much higher than, for example, primer-dimers, etc.

Hybridization Assay

Nucleic acid hybridization assays use probes to hybridize to the target nucleic acid, thereby allowing detection of the target nucleic acid. Non-limiting examples of hybridization assay include Northern blotting, Southern blotting, in situ hybridization, microarray analysis, and multiplexed hybridization-based assays.

In certain embodiments, the probes for hybridization assay are detectably labeled. In certain embodiments, the nucleic acid-based probes for hybridization assay are unlabeled. Such unlabeled probes can be immobilized on a solid support such as a microarray, and can hybridize to the target nucleic acid molecules which are detectably labeled.

In certain embodiments, hybridization assays can be performed by isolating the nucleic acids (e.g. RNA or DNA), separating the nucleic acids (e.g. by gel electrophoresis) followed by transfer of the separated nucleic acid on suitable membrane filters (e.g. nitrocellulose filters), where the probes hybridize to the target nucleic acids and allows detection. See, for example, Molecular Cloning: A Laboratory Manual, J. Sambrook et al., eds., 2nd edition, Cold Spring Harbor Laboratory Press, 1989, Chapter 7. The hybridization of the probe and the target nucleic acid can be detected or measured by methods known in the art. For example, autoradiographic detection of hybridization can be performed by exposing hybridized filters to photographic film.

In some embodiments, hybridization assays can be performed on microarrays. Microarrays provide a method for the simultaneous measurement of the levels of large numbers of target nucleic acid molecules. The target nucleic acids can be RNA, DNA, cDNA reverse transcribed from mRNA, or chromosomal DNA. The target nucleic acids can be allowed to hybridize to a microarray comprising a substrate having multiple immobilized nucleic acid probes arrayed at a density of up to several million probes per square centimeter of the substrate surface. The RNA or DNA in the sample is hybridized to complementary probes on the array and then detected by laser scanning. Hybridization intensities for each probe on the array are determined and converted to a quantitative value representing relative levels of the RNA or DNA. See, U.S. Pat. Nos. 6,040,138, 5,800,992 and 6,020,135, 6,033,860, and 6,344,316.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device. Useful microarrays are also commercially available, for example, microarrays from Affymetrix, from Nano String Technologies, QuantiGene 2.0 Multiplex Assay from Panomics.

Sequencing Methods

Sequencing methods useful in the measurement of the tyrosine expression level involves sequencing of the target nucleic acid. Any sequencing known in the art can be used to detect the expression level of the protein kinase of interest. In general, sequencing methods can be categorized to traditional or classical methods and high throughput sequencing (next generation sequencing). Traditional sequencing methods include Maxam-Gilbert sequencing (also known as chemical sequencing) and Sanger sequencing (also known as chain-termination methods).

High throughput sequencing, or next generation sequencing, by using methods distinguished from traditional methods, such as Sanger sequencing, is highly scalable and able to sequence the entire genome or transcriptome at once. High throughput sequencing involves sequencing-by-synthesis, sequencing-by-ligation, and ultra-deep sequencing (such as described in Marguiles et al., Nature 437 (7057): 376-80 (2005)). Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. Nos. 7,056,676, 8,802,368 and 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the ILLUMINA sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

In certain embodiments, the protein kinase expression level described herein is detected by whole transcriptome shotgun sequencing (RNA sequencing). The method of RNA sequencing has been described (see Wang Z, Gerstein M and Snyder M, Nature Review Genetics (2009) 10:57-63; Maher C A et al., Nature (2009) 458:97-101; Kukurba K & Montgomery S B, Cold Spring Harbor Protocols (2015) 2015(11): 951-969).

Immunoassay

Immunoassays used herein typically involves using antibodies that specifically bind to protein kinase protein. Such antibodies can be obtained using methods known in the art (see, e.g., Huse et al., Science (1989) 246:1275-1281; Ward et al, Nature (1989) 341:544-546), or can be obtained from commercial sources. Examples of immunoassays include, without limitation, Western blotting, enzyme-linked immunosorbent assay (ELISA), enzyme immunoassay (EIA), radioimmunoassay (RIA), immunoprecipitations, sandwich assays, competitive assays, immunofluorescent staining and imaging, immunohistochemistry (IHC), and fluorescent activating cell sorting (FACS). For a review of immunological and immunoassay procedures, see Basic and Clinical Immunology (Stites & Terr eds., 7^{th} ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology; Antibodies in Cell Biology, volume 37 (Asai, ed. 1993); Basic and Clinical Immunology (Stites & Terr, eds., 7^{th} ed. 1991).

Any of the assays and methods provided herein for the measurement of the kinase expression level can be adapted or optimized for use in automated and semi-automated systems, or point of care assay systems.

The kinase expression level described herein can be normalized using a proper method known in the art. For example, the kinase expression level can be normalized to a standard level of a standard marker, which can be predetermined, determined concurrently, or determined after a sample is obtained from the subject. The standard marker can be run in the same assay or can be a known standard marker from a previous assay. For another example, the protein kinase expression level can be normalized to an internal control which can be an internal marker, or an average level or a total level of a plurality of internal markers. For yet another example, in which the protein kinase expression is measured by mRNA level using high-throughput sequencing, the protein kinase expression level can be normalized to total hits of the sequencing assay.

Comparing with a Reference Level

In certain embodiments, the methods disclosed herein include a step of comparing the detected protein kinase expression level to a reference protein kinase level.

The term “reference protein kinase level” refers to a expression level of the protein kinase of interest that is representative of a reference sample. In certain embodiments, the reference sample is obtained from a healthy subject or tissue. In certain embodiments, the reference sample is a cancer or tumor tissue. In certain embodiments, the reference protein kinase level is obtained using the same or comparable measurement method or assay as used in the detection of the protein kinase expression level in the test sample.

In certain embodiments, the reference protein kinase level can be predetermined. For example, the reference protein kinase level can be calculated or generalized based on measurements of the protein kinase level in a collection of general cancer or tumor samples or tissues from a tumor of the same type, or from blood cancer. For another example, the reference proteinase kinase level can be based on statistics of the level of the protein kinase generally observed in an average cancer or tumor samples from a general cancer or tumor population.

In certain embodiments, the comparing step in the method provided herein involves determining the difference between the detected protein kinase expression level and the reference protein kinase level. The difference from the reference protein kinase level can be elevation or reduction.

In certain embodiments, the difference from the reference protein kinase level is further compared with a threshold. In certain embodiments, a threshold can be set by statistical methods, such that if the difference from the reference protein kinase level reaches the threshold, such difference can be considered statistically significant. Useful statistical analysis methods are described in L. D. Fisher & G. vanBelle, Biostatistics: A Methodology for the Health Sciences (Wiley-Interscience, N Y, 1993). Statistically significance can be determined based on confidence (“p”) values, which can be calculated using an unpaired 2-tailed t test. A p value less than or equal to, for example, 0.1, 0.05, 0.025, or 0.01 usually can be used to indicated statistical significance. Confidence intervals and p-values can be determined by methods well-known in the art. See, e.g., Dowdy and Wearden, Statistics for Research, John Wiley & Sons, New York, 1983.

Treatment with Protein Kinase Inhibitors

In one aspect, the present disclosure provides a method for treating cancer in a patient with a tyrosine kinase inhibitor. In certain embodiments, the method comprises: a) measuring the expression level of a first protein kinase in a sample obtained from the patient; b) comparing the expression level of the first protein kinase to a corresponding reference expression level; c) determining a likelihood of the patient being responsive to the tyrosine kinase inhibitor; and d) treating the patient whose expression level of the first protein kinase indicates that the patient will be responsive with the tyrosine kinase inhibitor.

In certain embodiments, the tyrosine kinase inhibitor is a multi-targeted tyrosine kinase inhibitor. Examples of multi-targeted tyrosine kinase inhibitors include Ponatinib, Cediranib, Sunitinib, Pazopanib, Imatinib, Sorafenib, Regorafenib, Anlotinib, Linifanib, Dovitinib, Bosutinib etc.

In some embodiments, the multi-targeted tyrosine kinase inhibitor preferentially inhibits a second tyrosine kinase different from the first tyrosine kinase. In yet another embodiment, the second protein kinase is KDR.

In certain embodiments, the tyrosine kinase inhibitor is an antibody, an antisense oligonucleotide or a compound. In some embodiments, the tyrosine kinase inhibitor is a compound of formula (I) or a pharmaceutically acceptable salt thereof

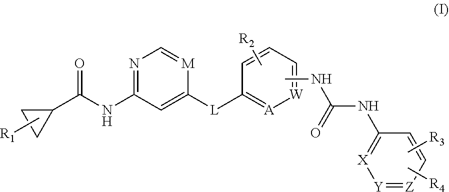

wherein:
R₁ is hydrogen, C₁₋₆ alkyl, C₁₋₆ haloalkyl, halo or cyano;
M is CH or N;
L is O, NH or N(CH₃);
A is CR₅ or N;
W is CR₆ or N;
R₂, R₅ and R₆ are independently hydrogen, C₁₋₆ alkyl, C₁₋₆ haloalkyl, halo, C₃₋₇ cycloalkyl or cyano;
X, Y, and Z are independently CH or N; and
R₃ and R₄ are independently hydrogen, halo, cyano, C₁₋₆ alkyl, C₁₋₆ haloalkyl, C₂₋₆ alkenyl, hydroxyl-C₁₋₆ alkyl, di-(C₁₋₆ alkylamino)-C₁₋₆ alkyl, amino, C₁₋₆ alkylamino, C₃₋₇ cycloalkylamino, di-(C₁₋₆ alkyl)amino, amino-C₁₋₆ alkylamino, C₁₋₆ alkoxy-C₁₋₆ alkylamino, C₁₋₆ alkoxycarbonyl-C₁₋₆ alkylamino, di-(C₁₋₆ alkoxy-C₁₋₆ alkyl)amino, aminocarbonyl, C₁₋₆ alkylaminocarbonyl, di-(C₁₋₆ alkyl)aminocarbonyl, C₃₋₇ cycloalkylaminocarbonyl, C₁₋₆ alkoxy, C₃₋₇ cycloalkoxy, hydroxyl-C₁₋₆ alkoxy, C₁₋₆ haloalkoxy, amino-C₁₋₆ alkyl, amino-C₁₋₆ alkoxy, C₁₋₆ alkylsulfonyl, C₂₋₆ alkenylsulfonyl, C₃₋₇ cycloalkylsulfonyl, heterocyclyl optionally substituted by B, aryl optionally substituted by B, heteroaryl optionally substituted by B, C₁₋₆ alkylsulfonylamino, C₂₋₆ alkenylsulfonylamino, C₃₋₇ cyclooalkylsulfonylamino, amido, C₁₋₆ alkylcarbonylamino, C₂₋₆ alkenylcarbonylamino, C₃₋₇ cyclooalkylcarbonylamino, C₁₋₆ alkoxycarbonylamino, C₃₋₇ cycloalkoxycarbonylamino, ureido, C₃₋₇ cycloalkyl, C₃₋₇ halocycloalkyl, heterocyclyl-oxy, piperidinylamino, N-methyl-piperidinyl-4-carbonyl, piperazinyl-C₁₋₆ alkyl, pyrrolylcarbonylamino, N-methyl-piperidinylcarbonylamino or heterocyclyl-C₁₋₆ alkoxy; or
R₃ and R₄ together form a 3 to 8-membered ring with the atoms in the aromatic ring to which they are attached; and B is hydrogen, C₁₋₆ alkyl, C₁₋₆ haloalkyl, halo, hydroxyl, aryl, amino, C₁₋₆ alkylamino, C₃₋₇ cycloalkylamino, di-(C₁₋₆ alkyl)amino, cyano, or C₃₋₇ cycloalkyl.

In certain embodiments,

R₁ is hydrogen;

M is N;

L is O;

A is CR₅;

W is CR₆;

R₂, R₅ and R₆ are independently hydrogen, C₁₋₆ alkyl, or halo;

X, Y, and Z are CH; and

R₃ and R₄ are independently hydrogen, halo, C₁₋₆ haloalkyl or C₁₋₆ haloalkoxy.

In one embodiment, the tyrosine kinase inhibitor has the structure selected from the following group:

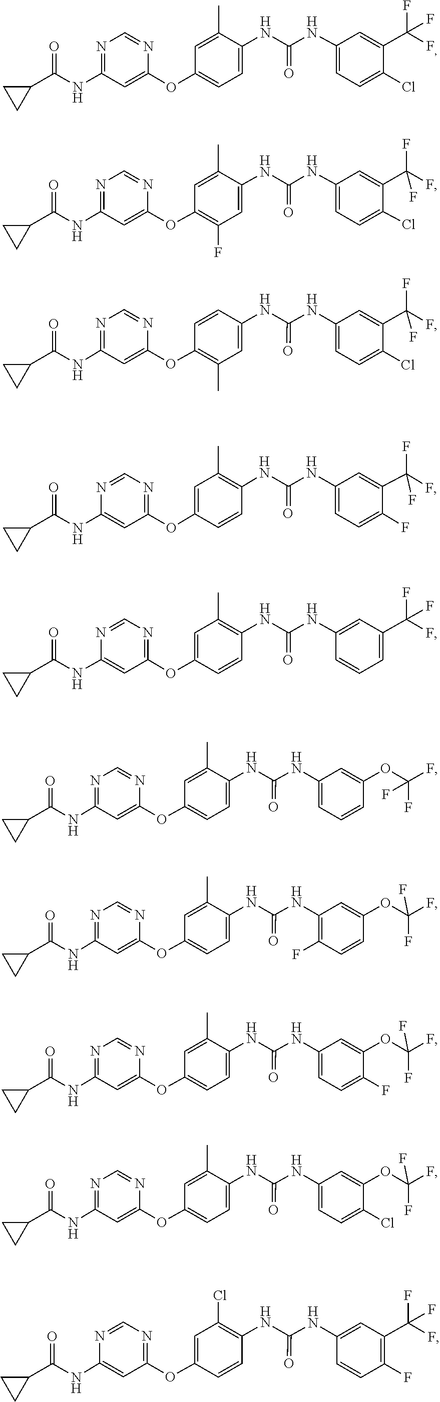

In one embodiment, the tyrosine kinase inhibitor is CBT-102, which has the following structures:

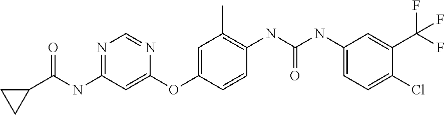 [SMILES: Cc1cc(Oc2cc(NC(=O)C3CC3)ncn2)ccc1NC(=O)Nc1ccc(Cl)c(C(F)(F)F)c1]

In certain embodiments, tyrosine kinase inhibitor can be formulated with a pharmaceutically acceptable carrier. The carrier, when present, can be blended with tyrosine kinase inhibitor in any suitable amounts, such as an amount of from 5% to 95% by weight of carrier, based on the total volume or weight of tyrosine kinase inhibitor and the carrier. In some embodiments, the amount of carrier can be in a range having a lower limit of any of 5%, 10%, 12%, 15%, 20%, 25%, 28%, 30%, 40%, 50%, 60%, 70% or 75%, and an upper limit, higher than the lower limit, of any of 20%, 22%, 25%, 28%, 30%, 40%, 50%, 60%, 70%, 75%, 80%, 85%, 90%, and 95%. The amount of carrier in a specific embodiment may be determined based on considerations of the specific dose form, relative amounts of tyrosine kinase inhibitor, the total weight of the composition including the carrier, the physical and chemical properties of the carrier, and other factors, as known to those of ordinary skill in the formulation art.

The tyrosine kinase inhibitor may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the tyrosine kinase inhibitor may be administered in conjunction with other treatments. The tyrosine kinase inhibitor may be encapsulated or otherwise protected against gastric or other secretions, if desired.

A suitable, non-limiting example of a dosage of the tyrosine kinase inhibitor disclosed herein is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. In some embodiments, the dosage of the tyrosine kinase inhibitor in human is about 400 mg/day given every 12 hours. In some embodiments, the dosage of the tyrosine kinase inhibitor in human ranges 300-500 mg/day, 100-600 mg/day or 25-1000 mg/day. The effective dose of tyrosine kinase inhibitor disclosed herein may be administered as two, three, four, five, six or more sub-doses, administered separately at appropriate intervals throughout the day.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

Materials and Methods

1. Kinase Profiling

For most assays, kinase-tagged T7 phage strains were grown in parallel in 24-well blocks in an E. coli host derived from the BL21 strain. The E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. Alternatively, some kinases were produced in HEK-293 cells. Streptavidin-coated magnetic beads were treated with biotinylated small molecule ligands of the kinases for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 40× stocks in 100% DMSO and directly diluted into the assay. All reactions were performed in polypropylene 384-well plates in a final volume of 0.04 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

The compound(s) were screened at the concentration(s) requested, and results for primary screen binding interactions are reported as percent control “% Ctrl”, where lower numbers indicate stronger hits in the matrix on the following page(s).

$$\% Ctrl Calculation = ( \frac{test compound signal - positive control signal}{negative control signal - positive control signal} ) \times 100$$

$$negative control = DMSO ( 100 \% Ctrl )$$

Selectivity Score or S-score is a quantitative measure of compound selectivity. It is calculated by dividing the number of kinases that compounds bind to by the total number of distinct kinases tested, excluding mutant variants.

S=Number of hits/Number of assays

This value can be calculated using % Ctrl as a potency threshold (below) and provides a quantitative method of describing compound selectivity to facilitate comparison of different compounds.

S(35)=(number of non-mutant kinases with % Ctrl<35)/(number of non-mutant kinases tested)

S(10)=(number of non-mutant kinases with % Ctrl<10)/(number of non-mutant kinases tested)

S(1)=(number of non-mutant kinases with % Ctrl<1)/(number of non-mutant kinases tested)

2. Kinase IC50 values of CBT-102

CBT-102 were tested against each of the selected kinases using the Eurofins standard KinaseProfder assays and following the relevant standard operating procedures. The required volume of the 50× stock of test compound (CBT-102) was added to the assay well, before a reaction mix containing the enzyme and substrate was added. The reaction was initiated by the addition of ATP at the selected concentration. There was no pre-incubation of the compound with the enzyme/substrate mix prior to ATP addition.

Data are handled using a custom-built in-house analysis software. Results are expressed as kinase activity remaining, as a percentage of the DMSO control. This is calculated using the following formula:

$$\frac{Mean of Sample Counts - Mean of Blank Counts}{Mean of Control Counts}$$

For IC50 determinations, data are analyzed using XLFit version 5.3 (ID Business Solutions).

3. CBT-102 In Vivo Efficacy in PDX Models

PDX tumor fragments, harvested from donor mice, were inoculated subcutaneously at the upper right dorsal flank into female BALB/c nude mice for tumor development. The randomization started when the mean tumor size reaches approximately 150 (100-200) mm³. The day of grouping will be denoted as Day 0 and dosing initiation were Day 1. Tumor volumes were measured two times per week in two dimensions using a caliper, and the volume were expressed in mm3 using the formula: “V=(L×W×W)/2, where V was tumor volume, L was tumor length (the longest tumor dimension) and W was tumor width (the longest tumor dimension perpendicular to L). PDX models included gastric cancer models, lung cancer models, colorectal cancer models, liver cancer, esophageal cancer, breast cancer etc.

4. Protein Kinase Expression in PDX Tumors

Tumors were harvested from PDX model, and the expression level of protein kinases were detected by RNAseq.

5. Evaluation of CBT-102 Effect on Proliferation of M-NFS-60 and RAW264.7 Syngeneic Cell Lines and Engineered BaF3 hCSFIR Cell Line

Two syngeneic cell lines M-NFS-60 and RAW264.7 and engineered BaF3 hCSFIR cell line were starved and then tested with CBT-102 and other test articles for 3 days in the temperature of 37° C., 5% CO₂ and 95% humidity. The cell viability was then detected using CTG assay. The software of GraphPad Prism was used to calculate IC50.

6. CBT-102 In Vivo Efficacy on Subcutaneous MC-38 Murine Colon Cancer Model

The MC-38 tumor cells were maintained in vitro with RPMI1640 medium supplemented with 10% fetal bovine serum at 37° C. in an atmosphere of 5% CO₂ in air. Each mouse was inoculated subcutaneously after shaving at the right flank region with MC-38 tumor cells (1×10e6) in 0.1 mL of PBS for tumor development. The randomization started when the mean tumor size reached approximately 100 mm³. Group 1 mice were treated vehicle and group 2 mice were treated 20 mg/kg CBT-102 at first 11 days and reduced to 10 mg/kg afterwards.

7. CD3/CD4/CD8/F4-80 IHC Test in MC-38 Tumor

MC38 tumor tissues were collected at the end of the in vivo efficacy trial. The tumor tissue was then embedded in paraffin. The expression of CD3/CD4/CD8/F4-80 were tested by immunohistochemistry stain.

### Example 2

This example illustrates the screening of protein kinases that can be effectively inhibited by CBT-102.

1. CBT-102 has Unique Kinase Profiler

The screen assay tested the inhibiting efficacy of CBT-102 against 403 candidate protein kinases. As illustrated in Table 1, CBT-102 demonstrated various efficacy (shown as % Control) in inhibiting the group of protein kinases. In particular, as shown in Table 2, among the 403 protein kinases being tested, CBT-102 demonstrated strong inhibition against 13 protein kinases (shown as S(1)) and moderate inhibition against 36 protein kinases (shown as S(10)).

TABLE 1

| | CBI-00005720 |
|---|---|
| Target | % Ctrl @ |
| Gene Symbol | 1000 nM |
| AAK1 | 96 |
| ABL1 (E255K)-phosphorylated | 92 |
| ABL1 (F317I)-nonphosphorylated | 70 |
| ABL1 (F317I)-phosphorylated | 95 |
| ABL1 (F317L)-nonphosphorylated | 16 |
| ABL1 (F317L)-phosphorylated | 52 |
| ABL1 (H396P)-nonphosphorylated | 20 |
| ABL1 (H396P)-phosphorylated | 83 |
| ABL1 (M351T)-phosphorylated | 65 |
| ABL1 (Q252H)-nonphosphorylated | 6 |
| ABL1 (Q252H)-phosphorylated | 82 |
| ABL1 (T315I)-nonphosphorylated | 2.3 |
| ABL1 (T315I)-phosphorylated | 51 |
| ABL1 (Y253F)-phosphorylated | 57 |
| ABL1-nonphosphorylated | 3.3 |
| ABL1-phosphorylated | 67 |
| ABL2 | 40 |
| ACVR1 | 95 |
| ACVR1B | 100 |
| ACVR2A | 100 |
| ACVR2B | 89 |
| ACVRL1 | 67 |
| ADCK3 | 100 |
| ADCK4 | 88 |
| AKT1 | 100 |
| AKT2 | 100 |
| AKT3 | 97 |
| ALK | 100 |
| ALK (C1156Y) | 97 |
| ALK (L1196M) | 94 |
| AMPK-alpha1 | 98 |
| AMPK-alpha2 | 100 |
| ANKK1 | 100 |
| ARK5 | 100 |
| ASK1 | 91 |
| ASK2 | 94 |
| AURKA | 86 |
| AURKB | 49 |
| AURKC | 33 |
| AXL | 56 |
| BIKE | 61 |
| BLK | 48 |
| BMPR1A | 54 |
| BMPR1B | 99 |
| BMPR2 | 82 |
| BMX | 90 |
| BRAF | 6.1 |
| BRAF (V600E) | 1.8 |
| BRK | 89 |
| BRSK1 | 95 |
| BRSK2 | 100 |
| BTK | 77 |
| BUB1 | 100 |
| CAMK1 | 74 |
| CAMK1B | 98 |
| CAMK1D | 81 |
| CAMK1G | 91 |
| CAMK2A | 88 |
| CAMK2B | 97 |
| CAMK2D | 93 |
| CAMK2G | 100 |
| CAMK4 | 100 |
| CAMKK1 | 88 |
| CAMKK2 | 92 |
| CASK | 98 |
| CDC2L1 | 36 |
| CDC2L2 | 22 |
| CDC2L5 | 73 |
| CDK11 | 0 |
| CDK2 | 69 |
| CDK3 | 90 |
| CDK4 | 100 |
| CDK4-cyclinD1 | 99 |
| CDK4-cyclinD3 | 100 |
| CDK5 | 92 |
| CDK7 | 25 |
| CDK8 | 14 |
| CDK9 | 46 |
| CDKL1 | 65 |
| CDKL2 | 0.45 |
| CDKL3 | 4.3 |
| CDKL5 | 97 |
| CHEK1 | 100 |
| CHEK2 | 100 |
| CIT | 87 |
| CLK1 | 98 |
| CLK2 | 100 |
| CLK3 | 100 |
| CLK4 | 95 |
| CSF1R | 0 |
| CSF1R-autoinhibited | 60 |
| CSK | 29 |
| CSNK1A1 | 99 |
| CSNK1A1L | 100 |
| CSNK1D | 77 |
| CSNK1E | 100 |
| CSNK1G1 | 100 |
| CSNK1G2 | 94 |
| CSNK1G3 | 96 |
| CSNK2A1 | 100 |
| CSNK2A2 | 100 |
| CTK | 74 |
| DAPK1 | 98 |
| DAPK2 | 95 |
| DAPK3 | 96 |
| DCAMKL1 | 100 |
| DCAMKL2 | 95 |
| DCAMKL3 | 100 |
| DDR1 | 0.3 |
| DDR2 | 1.1 |
| DLK | 89 |
| DMPK | 93 |
| DMPK2 | 100 |
| DRAK1 | 100 |
| DRAK2 | 100 |
| DYRK1A | 97 |
| DYRK1B | 100 |
| DYRK2 | 89 |
| EGFR | 76 |
| EGFR (E746-A750del) | 95 |
| EGFR (G719C) | 66 |
| EGFR (G719S) | 84 |
| EGFR (L747-E749del, A750P) | 80 |
| EGFR (L747-S752del, P753S) | 85 |
| EGFR (L747-T751del, Sins) | 88 |
| EGFR (L858R) | 87 |
| EGFR (L858R, T790M) | 86 |
| EGFR (L861Q) | 56 |
| EGFR (S752-I759del) | 91 |
| EGFR (T790M) | 94 |
| EIF2AK1 | 99 |
| EPHA1 | 45 |
| EPHA2 | 40 |
| EPHA3 | 33 |
| EPHA4 | 49 |
| EPHA5 | 52 |
| EPHA6 | 2.7 |
| EPHA7 | 4.1 |
| EPHA8 | 9.1 |
| EPHB1 | 38 |
| EPHB2 | 44 |
| EPHB3 | 82 |
| EPHB4 | 18 |
| EPHB6 | 14 |
| ERBB2 | 93 |
| ERBB3 | 100 |
| ERBB4 | 100 |
| ERK1 | 91 |
| ERK2 | 97 |
| ERK3 | 100 |
| ERK4 | 91 |
| ERK5 | 88 |
| ERK8 | 0.6 |
| ERN1 | 78 |
| FAK | 86 |
| FER | 73 |
| FES | 68 |
| FGFR1 | 61 |
| FGFR2 | 62 |
| FGFR3 | 87 |
| FGFR3 (G697C) | 100 |
| FGFR4 | 92 |
| FGR | 41 |
| FLT1 | 0.8 |
| FLT3 | 2.3 |
| FLT3 (D835H) | 42 |
| FLT3 (D835V) | 95 |
| FLT3 (D835Y) | 61 |
| FLT3 (ITD) | 18 |
| FLT3 (ITD, D835V) | 98 |
| FLT3 (ITD, F691L) | 86 |
| FLT3 (K663Q) | 3.9 |
| FLT3 (N841I) | 0 |
| FLT3 (R834Q) | 97 |
| FLT3-autoinhibited | 90 |
| FLT4 | 0.05 |
| FRK | 6.1 |
| FYN | 76 |
| GAK | 100 |
| GCN2 (Kin.Dom.2, S808G) | 100 |
| GRK1 | 100 |
| GRK2 | 100 |
| GRK3 | 97 |
| GRK4 | 100 |
| GRK7 | 100 |
| GSK3A | 18 |
| GSK3B | 42 |
| HASPIN | 81 |
| HCK | 54 |
| HIPK1 | 96 |
| HIPK2 | 90 |
| HIPK3 | 78 |
| HIPK4 | 11 |
| HPK1 | 23 |
| HUNK | 68 |
| ICK | 100 |
| IGF1R | 95 |
| IKK-alpha | 93 |
| IKK-beta | 92 |
| IKK-epsilon | 100 |
| INSR | 100 |
| INSRR | 100 |
| IRAK1 | 75 |
| IRAK3 | 100 |
| IRAK4 | 93 |
| ITK | 90 |
| JAK1 (JH1domain-catalytic) | 78 |
| JAK1 (JH2domain-pseudokinase) | 100 |
| JAK2 (JH1domain-catalytic) | 100 |
| JAK3 (JH1domain-catalytic) | 82 |
| JNK1 | 90 |
| JNK2 | 31 |
| JNK3 | 85 |
| KIT | 0 |
| KIT (A829P) | 16 |
| KIT (D816H) | 37 |
| KIT (D816V) | 18 |
| KIT (L576P) | 0 |
| KIT (V559D) | 0 |
| KIT (V559D, T670I) | 0.3 |
| KIT (V559D, V654A) | 2.8 |
| KIT-autoinhibited | 58 |
| LATS1 | 80 |
| LATS2 | 100 |
| LCK | 15 |
| LIMK1 | 74 |
| LIMK2 | 92 |
| LKB1 | 100 |
| LOK | 0.1 |
| LRRK2 | 73 |
| LRRK2 (G2019S) | 38 |
| LTK | 100 |
| LYN | 20 |
| LZK | 97 |
| MAK | 62 |
| MAP3K1 | 83 |
| MAP3K15 | 99 |
| MAP3K2 | 90 |
| MAP3K3 | 95 |
| MAP3K4 | 100 |
| MAP4K2 | 91 |
| MAP4K3 | 19 |
| MAP4K4 | 3.4 |
| MAP4K5 | 2.2 |
| MAPKAPK2 | 100 |
| MAPKAPK5 | 93 |
| MARK1 | 100 |
| MARK2 | 76 |
| MARK3 | 100 |
| MARK4 | 100 |
| MAST1 | 86 |
| MEK1 | 83 |
| MEK2 | 76 |
| MEK3 | 90 |
| MEK4 | 100 |
| MEK5 | 6.2 |
| MEK6 | 96 |
| MELK | 84 |
| MERTK | 23 |
| MET | 80 |
| MET (M1250T) | 84 |
| MET (Y1235D) | 79 |
| MINK | 35 |
| MKK7 | 66 |
| MKNK1 | 100 |
| MKNK2 | 83 |
| MLCK | 100 |
| MLK1 | 94 |
| MLK2 | 99 |
| MLK3 | 92 |
| MRCKA | 91 |
| MRCKB | 100 |
| MST1 | 100 |
| MST1R | 89 |
| MST2 | 92 |
| MST3 | 89 |
| MST4 | 100 |
| MTOR | 94 |
| MUSK | 47 |
| MYLK | 98 |
| MYLK2 | 98 |
| MYLK4 | 100 |
| MYO3A | 81 |
| MYO3B | 90 |
| NDR1 | 94 |
| NDR2 | 100 |
| NEK1 | 100 |
| NEK10 | 77 |
| NEK11 | 100 |
| NEK2 | 98 |
| NEK3 | 100 |
| NEK4 | 93 |
| NEK5 | 96 |
| NEK6 | 100 |
| NEK7 | 88 |
| NEK9 | 96 |
| NIK | 100 |
| NIM1 | 82 |
| NLK | 16 |
| OSR1 | 96 |
| p38-alpha | 0 |
| p38-beta | 0 |
| p38-delta | 8.6 |
| p38-gamma | 28 |
| PAK1 | 100 |
| PAK2 | 94 |
| PAK3 | 100 |
| PAK4 | 100 |
| PAK6 | 94 |
| PAK7 | 91 |
| PCTK1 | 88 |
| PCTK2 | 44 |
| PCTK3 | 84 |
| PDGFRA | 2.1 |
| PDGFRB | 0 |
| PDPK1 | 100 |
| PFCDPK1 (P.falciparum) | 53 |
| PFPK5 (P.falciparum) | 100 |
| PFTAIRE2 | 60 |
| PFTK1 | 34 |
| PHKG1 | 96 |
| PHKG2 | 87 |
| PIK3C2B | 100 |
| PIK3C2G | 100 |
| PIK3CA | 100 |
| PIK3CA (C420R) | 100 |
| PIK3CA (E542K) | 96 |
| PIK3CA (E545A) | 100 |
| PIK3CA (E545K) | 94 |
| PIK3CA (H1047L) | 100 |
| PIK3CA (H1047Y) | 100 |
| PIK3CA (I800L) | 91 |
| PIK3CA (M1043I) | 100 |
| PIK3CA (Q546K) | 100 |
| PIK3CB | 100 |
| PIK3CD | 100 |
| PIK3CG | 100 |
| PIK4CB | 100 |
| PIKFYVE | 91 |
| PIM1 | 95 |
| PIM2 | 100 |
| PIM3 | 98 |
| PIP5K1A | 100 |
| PIP5K1C | 93 |
| PIP5K2B | 99 |
| PIP5K2C | 85 |
| PKAC-alpha | 97 |
| PKAC-beta | 95 |
| PKMYT1 | 97 |
| PKN1 | 100 |
| PKN2 | 100 |
| PKNB (M.tuberculosis) | 100 |
| PLK1 | 95 |
| PLK2 | 93 |
| PLK3 | 97 |
| PLK4 | 98 |
| PRKCD | 100 |
| PRKCE | 99 |
| PRKCH | 100 |
| PRKCI | 74 |
| PRKCQ | 95 |
| PRKD1 | 100 |
| PRKD2 | 100 |
| PRKD3 | 91 |
| PRKG1 | 100 |
| PRKG2 | 91 |
| PRKR | 92 |
| PRKX | 93 |
| PRP4 | 95 |
| PYK2 | 93 |
| QSK | 91 |
| RAF1 | 3.4 |
| RET | 0 |
| RET (M918T) | 0.1 |
| RET (V804L) | 42 |
| RET (V804M) | 3.3 |
| RIOK1 | 54 |
| RIOK2 | 92 |
| RIOK3 | 65 |
| RIPK1 | 95 |
| RIPK2 | 28 |
| RIPK4 | 100 |
| RIPK5 | 100 |
| ROCK1 | 100 |
| ROCK2 | 100 |
| ROS1 | 63 |
| RPS6KA4 (Kin.Dom.1-N-terminal) | 100 |
| RPS6KA4 (Kin.Dom.2-C-terminal) | 100 |
| RPS6KA5 (Kin.Dom.1-N-terminal) | 100 |
| RPS6KA5 (Kin.Dom.2-C-terminal) | 100 |
| RSK1 (Kin.Dom.1-N-terminal) | 100 |
| RSK1 (Kin.Dom.2-C-terminal) | 94 |
| RSK2 (Kin.Dom.1-N-terminal) | 97 |
| RSK2 (Kin.Dom.2-C-terminal) | 83 |
| RSK3 (Kin.Dom.1-N-terminal) | 98 |
| RSK3 (Kin.Dom.2-C-terminal) | 92 |
| RSK4 (Kin.Dom.1-N-terminal) | 100 |
| RSK4 (Kin.Dom.2-C-terminal) | 73 |
| S6K1 | 99 |
| SBK1 | 92 |
| SGK | 100 |
| SgK110 | 98 |
| SGK2 | 92 |
| SGK3 | 82 |
| SIK | 95 |
| SIK2 | 100 |
| SLK | 1.4 |
| SNARK | 100 |
| SNRK | 100 |
| SRC | 76 |
| SRMS | 99 |
| SRPK1 | 56 |
| SRPK2 | 100 |
| SRPK3 | 100 |
| STK16 | 100 |
| STK33 | 63 |
| STK35 | 93 |
| STK36 | 2.5 |
| STK39 | 100 |
| SYK | 100 |
| TAK1 | 12 |
| TAOK1 | 49 |
| TAOK2 | 23 |
| TAOK3 | 9.6 |
| TBK1 | 98 |
| TEC | 92 |
| TESK1 | 100 |
| TGFBR1 | 100 |
| TGFBR2 | 56 |
| TIE1 | 20 |
| TIE2 | 16 |
| TLK1 | 100 |
| TLK2 | 100 |
| TNIK | 8.3 |
| TNK1 | 52 |
| TNK2 | 93 |
| TNNI3K | 31 |
| TRKA | 74 |
| TRKB | 71 |
| TRKC | 37 |
| TRPM6 | 99 |
| TSSK1B | 87 |
| TSSK3 | 100 |
| TTK | 61 |
| TXK | 100 |
| TYK2 (JH1domain-catalytic) | 5.6 |
| TYK2 (JH2domain-pseudokinase) | 100 |
| TYRO3 | 82 |
| ULK1 | 98 |
| ULK2 | 98 |
| ULK3 | 88 |
| VEGFR2 | 1.4 |
| VPS34 | 80 |
| VRK2 | 100 |
| WEE1 | 92 |
| WEE2 | 97 |
| WNK1 | 87 |
| WNK2 | 100 |
| WNK3 | 100 |
| WNK4 | 89 |
| YANK1 | 98 |
| YANK2 | 100 |
| YANK3 | 88 |
| YES | 55 |
| YSK1 | 100 |
| YSK4 | 9.6 |
| ZAK | 5.5 |
| ZAP70 | 90 |

TABLE 2

| | | | Number | | |
|---|---|---|---|---|---|
| | Select- | | of | Screening | |
| | ivity | Number | Non- | Concen- | Select- |
| Compound | Score | of | Mutant | tration | ivity |
| Name | Type | Hits | Kinases | (nM) | Score |
| CBI-00005720 | S (35) | 61 | 403 | 1000 | 0.151 |
| CBI-00005720 | S (10) | 36 | 403 | 1000 | 0.089 |
| CBI-00005720 | S (1) | 13 | 403 | 1000 | 0.032 |

2. Kinase IC50 Values of CBT-102

As illustrated in Table 3 below, the following protein kinases showed an IC50 of CBT-102 of less than 100 nM: CDKL2, cKit, c-RAF, DDR1, Flt1, Flt4, CSF1R, KDR, MAP4K5, PDGFRα, PTK5, Ret, SAPK2b, ZAK.

TABLE 3

| | CBT-102 |
|---|---|
| Kinase | IC₅₀ (nM) |
| Abl (h) | 1833 |
| Aurora-A (h) | 1768 |
| Aurora-B (h) | 525 |
| Aurora-C (h) | >10,000 |
| BRK (h) | >10,000 |
| B-Raf (h) | 348 |
| B-Raf (V599E) (h) | 115 |
| CDK2/cyclinE (h) | >10,000 |
| CDK4/cyclinD3 (h) | >10,000 |
| CDK6/cyclinD3 (h) | >10,000 |
| CDKL2 (h) | 37 |
| CDKL3 (h) | >10,000 |
| CHK1 (h) | >10,000 |
| cKit (h) | 1486 |
| cKit (D816V) (h) | >10,000 |
| cKit (V560G) (h) | 38 |
| cKit (V654A) (h) | 278 |
| CSK (h) | >10,000 |
| c-RAF (h) | 31 |
| cSRC (h) | 2496 |
| DDR1 (h) | 34 |
| DDR2 (h) | 263 |
| EGFR (h) | >10,000 |
| EphA1 (h) | 1113 |
| EphB2 (h) | 418 |
| EphB3 (h) | >10,000 |
| EphB4 (h) | 486 |
| ErbB2 (h) | >10,000 |
| ErbB4 (h) | >10,000 |
| FAK (h) | >10,000 |
| FGFR1 (h) | 410 |
| FGFR2 (h) | 596 |
| FGFR3 (h) | >10,000 |
| FGFR4 (h) | >10,000 |
| Fgr (h) | 1426 |
| Flt1 (h) | 21 |
| Flt3 (h) | 1086 |
| Flt4 (h) | 8 |
| Fms (h) (CSF1R) | 43 |
| IGF-1R (h) | >10,000 |
| IKKβ (h) | >10,000 |
| IR (h) | >10,000 |
| IRAK4 (h) | >10,000 |
| JAK2 (h) | >10,000 |
| KDR (h) | 25 |
| Lck (h) | 1341 |
| LOK (h) | 1087 |
| MAP4K4 (h) | 763 |
| MAP4K5 (h) | 24 |
| Met (h) | 2865 |
| MLK1 (h) | 6055 |
| MuSK (h) | 278 |
| NEK2 (h) | >10,000 |
| p70S6K (h) | >10,000 |
| PDGFRα (h) | 1594 |
| PDGFRα (V561D) (h) | 28 |
| PDGFRβ (h) | 1325 |
| Pim-1 (h) | >10,000 |
| PKA (h) | >10,000 |
| PKBα (h) | >10,000 |
| Plk1 (h) | >10,000 |
| PrKX (h) | >10,000 |
| PTK5 (h) | 52 |
| Ret (h) | 43 |
| Ret (V804M) (h) | 693 |
| RIPK2 (h) | 107 |
| SAPK2a (h) | 180 |
| SAPK2b (h) | 70 |
| SAPK3 (h) | 1197 |
| SAPK4 (h) | 228 |
| SGK3 (h) | >10,000 |
| SLK (h) | 879 |
| Syk (h) | >10,000 |
| TAK1 (h) | 481 |
| Tie2 (h) | 242 |
| TNIK (h) | 113 |
| TYK2 (h) | >10,000 |
| Wee1 (h) | >10,000 |
| ZAK (h) | 26 |

### Example 3

This example illustrates that the expression level of DDR1 is correlated with the efficacy of CBT-102.

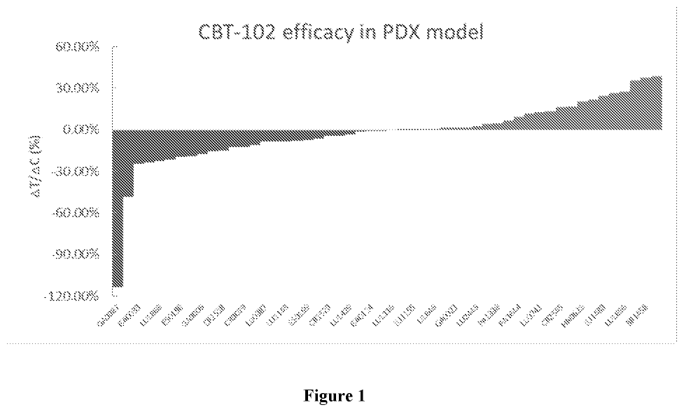

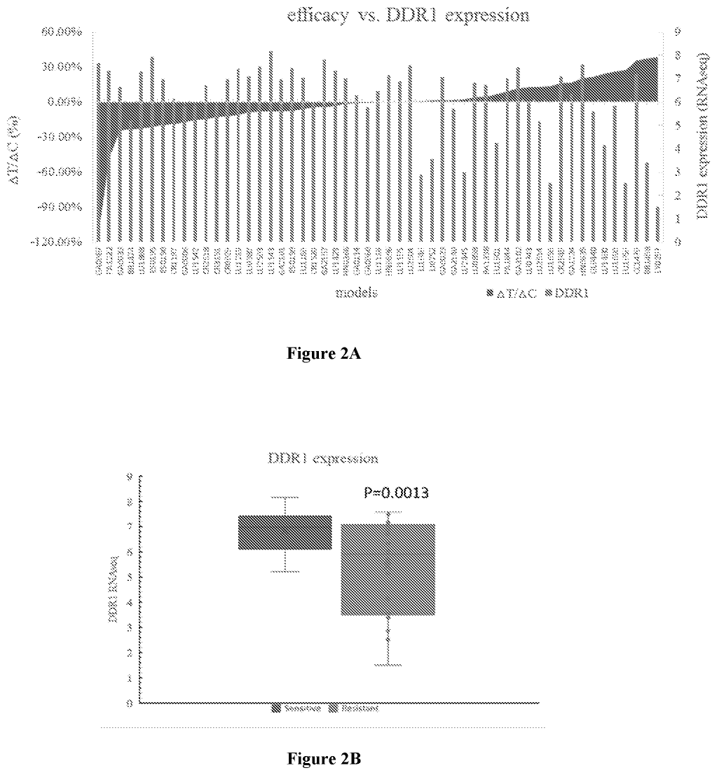

As shown in FIG. 1, CBT-102 demonstrated different in vivo efficacy in a group of PDX models. The expression level of DDR1 is then measured in these PDX models. The relationship between DDR1 expression level and the efficacy of CBT-102 in these PDX models was analyzed. As shown in FIGS. 2A and 2B, DDR1 expression level has a significant correlation with the efficacy of CBT-102 in the PDX models.

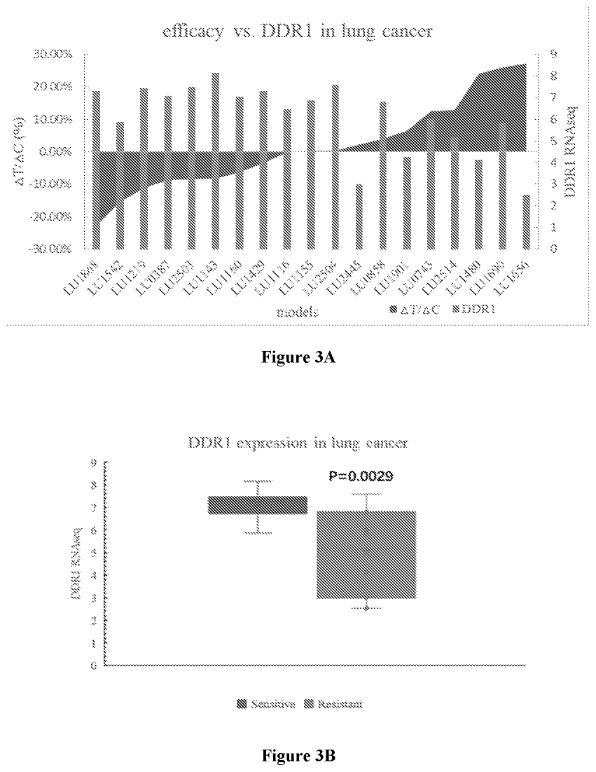

Of all the PDX models, the relationship between DDR1 expression and the efficacy of CBT-102 in lung cancer models was also analyzed. As shown in FIGS. 3A and 3B, DDR1 expression level has a significant correlation with the efficacy of CBT-102 in the lung cancer PDX models.

### Example 4

This example illustrates that CBT-102 inhibits cancer cell proliferation through the CSF-1/CSF-1R pathway.

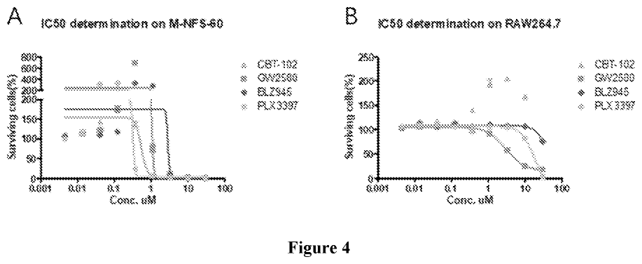

CSF-1 dependent mouse myeloid M-NFS-60 cells were employed as target cells while a CSF-1 independent cell line Raw 264.7 was used as a negative control. In addition to CBT-102, the study also included 3 CSF-1R inhibitors: GW2580, BLZ945 and Pexidartinib. As shown in Table 4 and FIGS. 4A and 4B, CBT-102, GW2580, BLZ945 and Pexidartinib effectively inhibited the proliferation of M-NFS-60. CBT-102, GW2580, BLZ945 and Pexidartinib have IC50 of 0.631/0.343, 1.145, 3.015 and 0.348 μM respectively. In contrast, the CSF-1 independent RAW264.7 cell displayed resistant to CBT-102 with IC50 of 22.85 μM, as well as the other two compounds BLC945 (IC50>30 μM) and Pexidartinib (IC50=16.36 μM). In engineered BaF3 hCSF1R cell lines, the IC50 value of CBT-102 was 0.588 μM compared to 1.333 μM of sulfatinib, a similar multi-kinase inhibitor, and 0.279 μM of GW2580, a more specific CSF1R kinase inhibitor. These results indicate that CBT-102

TABLE 4
IC50 of CBT-102 on 2 syngeneic cell lines

| | CBT-102 | | GW2580 | | BLZ945 | | PLX3397 | |
|---|---|---|---|---|---|---|---|---|
| | IC50 | Max | IC50 | Max | IC50 | Max | IC50 | Max |
| Cell line | (uM) | inh.(%) | (uM) | inh.(%) | (uM) | inh.(%) | (uM) | inh.(%) |
| M-NFS-60 | 0.631/ | 99.44%/ | 1.145 | 99.02% | 3.015 | 99.08% | 0.348 | 99.32% |
| | 0.343* | 99.78%* | | | | | | |
| RAW264.7 | 22.85 | 95.44% | 4.056 | 81.91% | >30 | 24.65% | 16.36 | 95.16% |

2. CBT-102 Inhibited the Growth of MC-38 Tumor

TABLE 5

| | | Tumor | | | |
|---|---|---|---|---|---|
| | Treatment | Size (mm³)^{a} | TGI | T/C | P |
| Group | Description | on day34 | (%) | (%) | value |
| 1 | Vehicle | 2262.85 ± 167.81 | — | — | — |
| 2 | CBT-102 | 454.63 ± 60.72 | 79.91 | 20.09 | <0.001 |

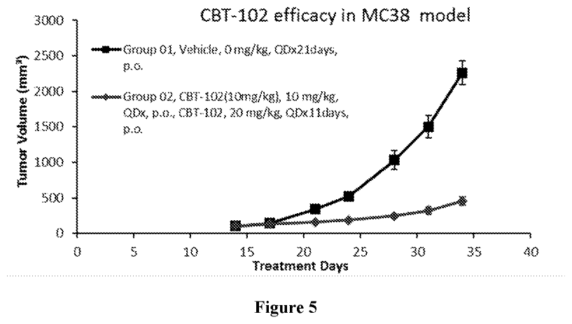

The treatment was performed on Day 14. Treatment with CBT-102 at 20 mg/kg (10 mg/kg) produced anti-tumor efficacy, the tumor sizes were 454.63 mm³ on Day34 (PG-D21) after tumor inoculation. (TGI: 79.91%, p<0.001 compared with the vehicle treated group) (FIG. 5).

3. CBT-102 Reduced F4-80 IHC Score in MC-38 Tumor

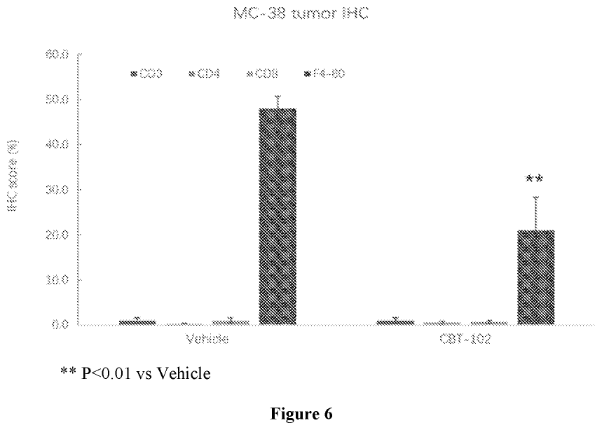

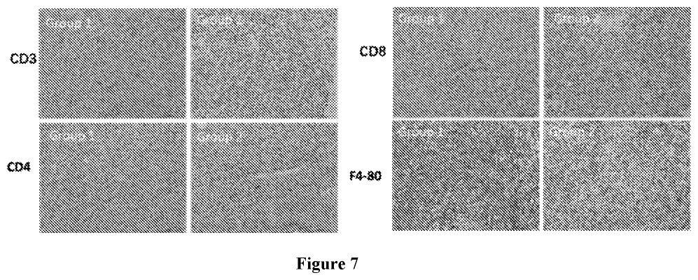

After treatment with CBT-102, F4-80 IHC score (%) in MC-38 tumor tissue was significantly reduced. F4-80 is the surface marker of macrophages. The results showed that CBT-102 had an effect on macrophages in tumor tissue. CBT-102 had no significant effect on T cell surface markers CD3, CD4, and CD8 in MC-38 tumors compared with vehicle group (FIGS. 6 and 7).

1. A method for treating cancer in a patient with a tyrosine kinase inhibitor, the method comprising:
a) measuring the expression level of a first protein kinase in a sample obtained from the patient;
b) comparing the expression level of the first protein kinase to a corresponding reference expression level;
c) determining a likelihood of the patient being responsive to the tyrosine kinase inhibitor; and
d) treating the patient whose expression level of the first protein kinase indicates that the patient will be responsive with the tyrosine kinase inhibitor,
wherein the tyrosine kinase inhibitor is a compound of formula (I) or a pharmaceutically acceptable salt thereof 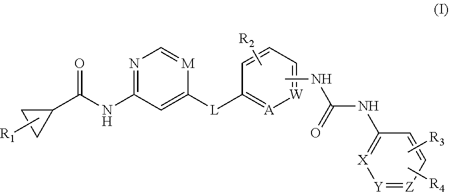
wherein:
R1 is hydrogen, C1-6 alkyl, C1-6 haloalkyl halo or cyano;
M is CH or N;
L is O, NH or N(CH3);
A is CR5 or N;
W is CR6 or N;
R2, R5 and R6 are independently hydrogen, C1-6 alkyl, C1-6 haloalkyl, halo, C3-7 cycloalkyl or cyano;
X, Y, and Z are independently CH or N; and
R3 and R4 are independently hydrogen, halo, cyano, C1-6 alkyl, C1-6 haloalkyl, C2-6 alkenyl, hydroxyl-C1-6 alkyl, di-(C1-6 alkylamino)-C1-6 alkyl, amino, C1-6 alkylamino, C3-7 cycloalkylamino, di-(C1-6 alkyl)amino, amino-C1-6 alkylamino, C1-6 alkoxy-C1-6 alkylamino, C1-6 alkoxycarbonyl-C1-6 alkylamino, di-(C1-6 alkoxy-C1-6 alkyl)amino, aminocarbonyl, C1-6 alkylaminocarbonyl, di-(C1-6 alkyl)aminocarbonyl, C3-7 cycloalkylaminocarbonyl, C1-6 alkoxy, C3-7 cycloalkoxy, hydroxyl-C1-6 alkoxy, C1-6 haloalkoxy, amino-C1-6 alkyl, amino-C1-6 alkoxy, C1-6 alkylsulfonyl, C2-6 alkenylsulfonyl, C3-7 cycloalkylsulfonyl, heterocyclyl optionally substituted by B, aryl optionally substituted by B, heteroaryl optionally substituted by B, C1-6 alkylsulfonylamino, C2-6 alkenylsulfonylamino, C3-7 cyclooalkylsulfonylamino, amido, C1-6 alkylcarbonylamino, C2-6 alkenylcarbonylamino, C3-7 cyclooalkylcarbonylamino, C1-6 alkoxycarbonylamino, C3-7 cycloalkoxycarbonylamino, ureido, C3-7 cycloalkyl, C3-7 halocycloalkyl, heterocyclyloxy, piperidinylamino, N-methyl-piperidinyl-4-carbonyl, piperazinyl-C1-6 alkyl, pyrrolylcarbonylamino, N-methyl-piperidinylcarbonylamino or heterocyclyl-C1-6 alkoxy; or
R3 and R4 together form a 3 to 8-membered ring with the atoms in the aromatic ring to which they are attached; and
B is hydrogen, C1-6 alkyl, C1-6 haloalkyl, halo, hydroxyl, aryl, amino, C1-6 alkylamino, C3-7 cycloalkylamino, di-(C1-6 alkyl)amino, cyano, or C3-7 cycloalkyl.

2. The method of claim 1, wherein the first protein kinase is selected from the group consisting of DDR1, CSF1R, CDKL2, cKit, c-RAF, Flt1, Flt4, KDR, MAP4K5, PDGFRα, PTK5, Ret, SAPK2b and ZAK.

3. The method of claim 1, wherein the first protein kinase is DDR1 or CSF1R.

4-9. (canceled)

10. The method of claim 1, wherein the tyrosine kinase inhibitor has the following structures:

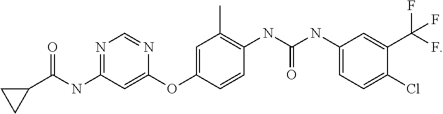 [SMILES: Cc1cc(Oc2cc(NC(=O)C3CC3)ncn2)ccc1NC(=O)Nc1ccc(Cl)c(C(F)(F)F)c1]

11. The method of claim 1, wherein the expression level of the first protein kinase is an RNA level, a protein level or a protein activation level.

12. The method of claim 1, wherein the expression level of the first protein kinase is measured by an amplification assay, a hybridization assay, a sequencing assay, an array, a western-blot, an immunohistochemistry or an ELISA.

13. The method of claim 1, wherein the cancer is selected from the group consisting of gastric cancer, a lung cancer, esophageal cancer, a melanoma, a renal cancer, a liver cancer, a myeloma, a prostate cancer, a breast cancer, a colorectal cancer, a pancreatic cancer, a thyroid cancer, a hematological cancer, a leukemia and a non-Hodgkin's lymphoma.

14. The method of claim 1, wherein the cancer is gastric cancer, lung cancer, colorectal cancer, liver cancer, esophageal cancer, a renal cancer or breast cancer.

15. A method for continuing a cancer therapy in a patient comprising:
a) treating the patient with a tyrosine kinase inhibitor;
b) obtaining a tumor sample from the patient;
c) measuring the expression level of a first protein kinase;
d) comparing the expression level of the first protein kinase to a corresponding reference expression level;
e) determining a likelihood of the patient being responsive to the tyrosine kinase inhibitor; and
f) continuing treatment of the cancer when the expression level of the first protein kinase in the tumor sample demonstrates responsiveness,
wherein the tyrosine kinase inhibitor is a compound of formula (I) or a pharmaceutically acceptable salt thereof 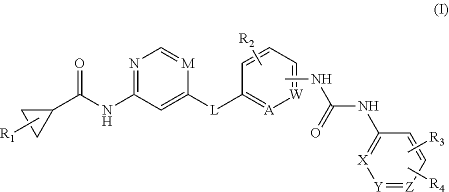
wherein:
R1 is hydrogen, C1-6 alkyl, C1-6 haloalkyl, halo or cyano;
M is CH or N;
L is O; NH or N(CH3);
A is CR5 or N;
W is CR6 or N;
R2, R5 and R6 are independently hydrogen, C1-6 alkyl, C1-6 haloalkyl, halo, C3-7 cycloalkyl or cyano;
X, Y, and Z are independently CH or N; and
R3 and R4 are independently hydrogen, halo, cyano, C1-6 alkyl, C1-6 haloalkyl, C2-6 alkenyl, hydroxyl-C1-6 alkyl, di-(C1-6 alkylamino)-C1-6 alkyl, amino, C1-6 alkylamino, C3-7 cycloalkylamino, di-(C1-6 alkyl)amino, amino-C1-6 alkylamino, C1-6 alkoxy-C1-6 alkylamino, C1-6 alkoxycarbonyl-C1-6 alkylamino, di-(C1-6 alkoxy-C1-6 alkyl)amino, aminocarbonyl, C1-6 alkylaminocarbonyl, di-(C1-6 alkyl)aminocarbonyl, C3-7 cycloalkylaminocarbonyl, C1-6 alkoxy, C3-7 cycloalkoxy, hydroxyl-C1-6 alkoxy, C1-6 haloalkoxy, amino-C1-6 alkyl, amino-C1-6 alkoxy, C1-6 alkylsulfonyl, C2-6 alkenylsulfonyl, C3-7 cycloalkylsulfonyl, heterocyclyl optionally substituted by B, aryl optionally substituted by B, heteroaryl optionally substituted by B, C1-6 alkylsulfonylamino, C2-6 alkenylsulfonylamino, C3-7 cyclooalkylsulfonylamino, amido, C1-6 alkylcarbonylamino, C2-6 alkenylcarbonylamino, C3-7 cyclooalkylcarbonylamino, C1-6 alkoxycarbonylamino, C3-7 cycloalkoxycarbonylamino, ureido, C3-7 cycloalkyl, C3-7 halocycloalkyl, heterocyclyl-oxy, piperidinylamino, N-methyl-piperidinyl-4-carbonyl, piperazinyl-C1-6 alkyl, pyrrolylcarbonylamino, N-methyl-piperidinylcarbonylamino or heterocyclyl-C1-6 alkoxy: or
R3 and R4 together form a 3 to 8-membered ring with the atoms in the aromatic ring to which they are attached; and
B is hydrogen, C1-6 alkyl, C1-6 haloalkyl, halo, hydroxyl, aryl, amino, C1-6 alkylamino, C3-7 cycloalkylamino, di-(C1-6 alkyl)amino, cyano, or C3-7 cycloalkyl.

16. The method of claim 15, wherein the first protein kinase is selected from the group consisting of DDR1, CSF1R, CDKL2, cKit, c-RAF, Flt1, Flt4, KDR, MAP4K5, PDGFRa, PTK5, Ret, SAPK2b and ZAK.

17. The method of claim 15, wherein the first protein kinase is DDR1 or CSF1R.

18-21. (canceled)

22. The method of claim 15, wherein the expression level of the first protein kinase is an RNA level, a protein level or a protein activation level.

23. The method of claim 15, wherein the expression level of the first protein kinase is measured by an amplification assay, a hybridization assay, a sequencing assay or an array, a western-blot, an immunohistochemistry or an ELISA.

24. The method of claim 15, wherein the cancer is selected from the groups consisting of gastric cancer, a lung cancer, esophageal cancer, a melanoma, a renal cancer, a liver cancer, a myeloma, a prostate cancer, a breast cancer, a colorectal cancer, a pancreatic cancer, a thyroid cancer, a hematological cancer, a leukemia and a non-Hodgkin's lymphoma.

25. The method of claim 15, wherein the tumor sample is a tissue sample or a blood sample.

26-29. (canceled)

30. The method of claim 15, wherein the tyrosine kinase inhibitor has the following structures: 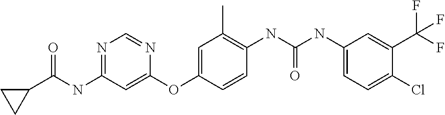 [SMILES: Cc1cc(Oc2cc(NC(=O)C3CC3)ncn2)ccc1NC(=O)Nc1ccc(Cl)c(C(F)(F)F)c1]


SEQUENCE LISTING (4 sequences)

SEQ ID NO: 1 - DNA, 3840 bp, Homo sapiens
gtcttcccctcgtgggccctgagcgggactgcagccagccccctggggcgccagctttggaggcccccgacagctgctctcgggagccgcctcccgacacccgagccccgccggcgcctcccgctcccggctcccggctcctggctccctccgcctcccccgcccctcgccccgccgccgaagaggccccgctcccgggtcggacgcctgggtctgccgggaagagcgatgagaggtgtctgaaggtggctattcactgagcgatggggttggacttgaaggaatgccaagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggcagctcaccgacgagcaggtcatcgagaacgcgggggagttcttccgggaccagggccggcaggtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaaaaaa

SEQ ID NO: 2 - protein, 876 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 3 - DNA, 4006 bp, Homo sapiens
gaagggcagacagagtgtccaaaagcgtgagagcacgaagtgaggagaaggtggagaagagagaagaggaagaggaagaggaagagaggaagcggagggaactgcggccaggctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgcagtcaaaaaaaaaaaaaaaaaa

SEQ ID NO: 4 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC